SEQUENCE LISTING (298 sequences)

SEQ ID NO: 1 - DNA, 17 bp, Artificial Sequence
  M13 forward primer
gtaaaacgacggccagt

SEQ ID NO: 2 - DNA, 20 bp, Artificial Sequence
  T7 universal primer
taatacgactcactataggg

SEQ ID NO: 3 - DNA, 38 bp, Artificial Sequence
  TRB-V-C-1
gtaaaacgacggccagttcgcttctcacctgaatgccc

SEQ ID NO: 4 - DNA, 39 bp, Artificial Sequence
  TRB-V-A-1
gtaaaacgacggccagtctcagttgaaaggcctgatgga

SEQ ID NO: 5 - DNA, 39 bp, Artificial Sequence
  TRB-V-X-1
gtaaaacgacggccagtggaagcatccctgatcgattct

SEQ ID NO: 6 - DNA, 39 bp, Artificial Sequence
  TRB-V-AA-1
gtaaaacgacggccagttcagctaagtgcctcccaaatt

SEQ ID NO: 7 - DNA, 39 bp, Artificial Sequence
  TRB-V-B-1
gtaaaacgacggccagtagttccaaatcgcttctcacct

SEQ ID NO: 8 - DNA, 39 bp, Artificial Sequence
  TRB-V-F-1
gtaaaacgacggccagtttccctaatcgattctcagggc

SEQ ID NO: 9 - DNA, 39 bp, Artificial Sequence
  TRB-V-J-1
gtaaaacgacggccagttacaactgccaaaggagaggtc

SEQ ID NO: 10 - DNA, 39 bp, Artificial Sequence
  TRB-V-L-1
gtaaaacgacggccagttaaaggagaagtcccgaatggc

SEQ ID NO: 11 - DNA, 39 bp, Artificial Sequence
  TRB-V-M-1
gtaaaacgacggccagtggagaagttcccaatggctaca

SEQ ID NO: 12 - DNA, 39 bp, Artificial Sequence
  TRB-V-S-1
gtaaaacgacggccagtataaaggagaagtccccgatgg

SEQ ID NO: 13 - DNA, 39 bp, Artificial Sequence
  TRB-V-W-1
gtaaaacgacggccagtctctagatgattcggggatgcc

SEQ ID NO: 14 - DNA, 39 bp, Artificial Sequence
  TRB-V-Z-1
gtaaaacgacggccagttgaagcagacacccctgataac

SEQ ID NO: 15 - DNA, 39 bp, Artificial Sequence
  TRB-V-AE-1
gtaaaacgacggccagttgagcgatttttagcccaatgc

SEQ ID NO: 16 - DNA, 40 bp, Artificial Sequence
  TRB-V-AG-1
gtaaaacgacggccagtacaaaggagagatctctgatgga

SEQ ID NO: 17 - DNA, 42 bp, Artificial Sequence
  TRB-J-A-1
taatacgactcactatagggctacaactgtgagtctggtgcc

SEQ ID NO: 18 - DNA, 41 bp, Artificial Sequence
  TRB-J-B-1
taatacgactcactatagggctacaacggttaacctggtcc

SEQ ID NO: 19 - DNA, 42 bp, Artificial Sequence
  TRB-J-C-1
taatacgactcactatagggtacaacagtgagccaacttccc

SEQ ID NO: 20 - DNA, 41 bp, Artificial Sequence
  TRB-J-D-1
taatacgactcactatagggcaagacagagagctgggttcc

SEQ ID NO: 21 - DNA, 42 bp, Artificial Sequence
  TRB-J-E-1
taatacgactcactatagggctaggatggagagtcgagtccc

SEQ ID NO: 22 - DNA, 40 bp, Artificial Sequence
  TRB-J-F-1
taatacgactcactatagggctgtcacagtgagcctggtc

SEQ ID NO: 23 - DNA, 42 bp, Artificial Sequence
  TRB-J-G-1
taatacgactcactatagggccttcttacctagcacggtgag

SEQ ID NO: 24 - DNA, 42 bp, Artificial Sequence
  TRB-J-H-1
taatacgactcactatagggttacccagtacggtcagcctag

SEQ ID NO: 25 - DNA, 40 bp, Artificial Sequence
  TRB-J-I-1
taatacgactcactatagggcttaccgagcactgtcagcc

SEQ ID NO: 26 - DNA, 40 bp, Artificial Sequence
  TRB-J-J-1
taatacgactcactatagggcttacccagcactgagagcc

SEQ ID NO: 27 - DNA, 39 bp, Artificial Sequence
  TRB-J-K-1
taatacgactcactatagggtcaccgagcaccaggagcc

SEQ ID NO: 28 - DNA, 42 bp, Artificial Sequence
  TRB-J-N-1
taatacgactcactataggggaatctcacctgtgaccgtgag

SEQ ID NO: 29 - DNA, 38 bp, Artificial Sequence
  TRB-V-D-1
gtaaaacgacggccagtggaaacttccctggtcgattc

SEQ ID NO: 30 - DNA, 39 bp, Artificial Sequence
  TRB-V-N-1
gtaaaacgacggccagtcaacgatcggttctttgcagtc

SEQ ID NO: 31 - DNA, 39 bp, Artificial Sequence
  TRB-V-O-1
gtaaaacgacggccagttaaatcagggctgctcagtgat

SEQ ID NO: 32 - DNA, 39 bp, Artificial Sequence
  TRB-V-P-1
gtaaaacgacggccagtcagtgatcggttctctgcagag

SEQ ID NO: 33 - DNA, 39 bp, Artificial Sequence
  TRB-V-R-1
gtaaaacgacggccagtcttgaacgattctccgcacaac

SEQ ID NO: 34 - DNA, 39 bp, Artificial Sequence
  TRB-V-V-1
gtaaaacgacggccagtccgaggatcgattctcagctaa

SEQ ID NO: 35 - DNA, 39 bp, Artificial Sequence
  TRB-V-AB-1
gtaaaacgacggccagtgccaaaggaacgattttctgct

SEQ ID NO: 36 - DNA, 39 bp, Artificial Sequence
  TRB-V-AI-1
gtaaaacgacggccagtagggagatgttcctgaagggta

SEQ ID NO: 37 - DNA, 39 bp, Artificial Sequence
  TRB-V-AJ-1
gtaaaacgacggccagtcctgaggggtacagtgtctcta

SEQ ID NO: 38 - DNA, 39 bp, Artificial Sequence
  TRB-V-AL-1
gtaaaacgacggccagtcagaatctctcagcctccagac

SEQ ID NO: 39 - DNA, 39 bp, Artificial Sequence
  TRB-V-E-1
gtaaaacgacggccagtacttccctgatcgattctcagc

SEQ ID NO: 40 - DNA, 39 bp, Artificial Sequence
  TRB-V-H-1
gtaaaacgacggccagtctcaggtcaccagttccctaac

SEQ ID NO: 41 - DNA, 39 bp, Artificial Sequence
  TRB-V-I-1
gtaaaacgacggccagtcctagattttcaggtcgccagt

SEQ ID NO: 42 - DNA, 39 bp, Artificial Sequence
  TRB-V-Q-1
gtaaaacgacggccagtctcaactagacaaatcggggct

SEQ ID NO: 43 - DNA, 39 bp, Artificial Sequence
  TRB-V-U-1
gtaaaacgacggccagtatcgattttctgcagagaggct

SEQ ID NO: 44 - DNA, 39 bp, Artificial Sequence
  TRB-V-Y-1
gtaaaacgacggccagtcggtatgcccaacaatcgattc

SEQ ID NO: 45 - DNA, 38 bp, Artificial Sequence
  TRB-V-AC-1
gtaaaacgacggccagtctgaagggtacagcgtctctc

SEQ ID NO: 46 - DNA, 39 bp, Artificial Sequence
  TRB-V-AH-1
gtaaaacgacggccagttcctctgagtcaacagtctcca

SEQ ID NO: 47 - DNA, 39 bp, Artificial Sequence
  TRB-V-AK-1
gtaaaacgacggccagtctgaggccacatatgagagtgg

SEQ ID NO: 48 - DNA, 41 bp, Artificial Sequence
  TRB-J-L-1
taatacgactcactataggggaaaactcacccagcacggtc

SEQ ID NO: 49 - DNA, 39 bp, Artificial Sequence
  TRB-J-M-1
taatacgactcactatagggtcacccagcacggtcagcc

SEQ ID NO: 50 - DNA, 39 bp, Artificial Sequence
  TRB-V-G-1
gtaaaacgacggccagtgattctcaggtctccagttccc

SEQ ID NO: 51 - DNA, 39 bp, Artificial Sequence
  TRB-V-K-1
gtaaaacgacggccagttaccactggcaaaggagaagtc

SEQ ID NO: 52 - DNA, 39 bp, Artificial Sequence
  TRB-V-T-1
gtaaaacgacggccagtcaaaggagaagtctcagatggc

SEQ ID NO: 53 - DNA, 39 bp, Artificial Sequence
  TRB-V-AD-1
gtaaaacgacggccagttttctcatcaaccatgcaagcc

SEQ ID NO: 54 - DNA, 39 bp, Artificial Sequence
  TRB-V-AF-1
gtaaaacgacggccagtggagatgcacaagaagcgattc

SEQ ID NO: 55 - DNA, 42 bp, Artificial Sequence; identical to SEQ ID NO: 17
  TRB-J-A-1

SEQ ID NO: 56 - DNA, 41 bp, Artificial Sequence; identical to SEQ ID NO: 18
  TRB-J-B-1

SEQ ID NO: 57 - DNA, 42 bp, Artificial Sequence; identical to SEQ ID NO: 19
  TRB-J-C-1

SEQ ID NO: 58 - DNA, 41 bp, Artificial Sequence; identical to SEQ ID NO: 20
  TRB-J-D-1

SEQ ID NO: 59 - DNA, 42 bp, Artificial Sequence; identical to SEQ ID NO: 21
  TRB-J-E-1

SEQ ID NO: 60 - DNA, 40 bp, Artificial Sequence; identical to SEQ ID NO: 22
  TRB-J-F-1

SEQ ID NO: 61 - DNA, 42 bp, Artificial Sequence; identical to SEQ ID NO: 23
  TRB-J-G-1

SEQ ID NO: 62 - DNA, 42 bp, Artificial Sequence; identical to SEQ ID NO: 24
  TRB-J-H-1

SEQ ID NO: 63 - DNA, 40 bp, Artificial Sequence; identical to SEQ ID NO: 25
  TRB-J-I-1

SEQ ID NO: 64 - DNA, 40 bp, Artificial Sequence; identical to SEQ ID NO: 26
  TRB-J-J-1

SEQ ID NO: 65 - DNA, 39 bp, Artificial Sequence; identical to SEQ ID NO: 27
  TRB-J-K-1

SEQ ID NO: 66 - DNA, 42 bp, Artificial Sequence; identical to SEQ ID NO: 28
  TRB-J-N-1

SEQ ID NO: 67 - DNA, 37 bp, Artificial Sequence
  TRB-D-A-1
gtaaaacgacggccagtcctccactcccctcaaagga

SEQ ID NO: 68 - DNA, 38 bp, Artificial Sequence
  TRB-D-B-1
gtaaaacgacggccagtcagactaacctctgccacctg

SEQ ID NO: 69 - DNA, 41 bp, Artificial Sequence; identical to SEQ ID NO: 48
  TRB-J-L-1

SEQ ID NO: 70 - DNA, 39 bp, Artificial Sequence; identical to SEQ ID NO: 49
  TRB-J-M-1

SEQ ID NO: 71 - DNA, 43 bp, Artificial Sequence
  TRG-V-E-1
gtaaaacgacggccagtcaagcatgaggaggagctggaaattg

SEQ ID NO: 72 - DNA, 38 bp, Artificial Sequence
  TRG-V-F-1
gtaaaacgacggccagtacgtctacatccactctcacc

SEQ ID NO: 73 - DNA, 40 bp, Artificial Sequence
  TRG-V-A-1
gtaaaacgacggccagtgcacaaggaacaacttgagattg

SEQ ID NO: 74 - DNA, 43 bp, Artificial Sequence
  TRG-V-B-1
gtaaaacgacggccagttggaagcacaaggaagaacttgagaa

SEQ ID NO: 75 - DNA, 39 bp, Artificial Sequence
  TRG-V-C-1
gtaaaacgacggccagtgcacagggaagagccttaaatt

SEQ ID NO: 76 - DNA, 38 bp, Artificial Sequence
  TRG-V-D-1
gtaaaacgacggccagtcaggaggtggagctggatatt

SEQ ID NO: 77 - DNA, 42 bp, Artificial Sequence
  TRG-V-G-1
gtaaaacgacggccagtctctcacttcaatccttaccatcaa

SEQ ID NO: 78 - DNA, 51 bp, Artificial Sequence
  TRG-V-H-1
gtaaaacgacggccagtgctcacacttccacttccactttgaaaataaagt

SEQ ID NO: 79 - DNA, 41 bp, Artificial Sequence
  TRG-J-A-1
taatacgactcactatagggagtgttgttccactgccaaag

SEQ ID NO: 80 - DNA, 41 bp, Artificial Sequence
  TRG-J-B-1
taatacgactcactataggggttccgggaccaaataccttg

SEQ ID NO: 81 - DNA, 43 bp, Artificial Sequence
  TRG-J-C-1
taatacgactcactataggggagcttagtcccttcagcaaata

SEQ ID NO: 82 - DNA, 40 bp, Artificial Sequence
  TRG-J-D-1
taatacgactcactatagggcctagtcccttttgcaaacg

SEQ ID NO: 83 - DNA, 41 bp, Artificial Sequence
  TRD-V-A-1
gtaaaacgacggccagtgaatgcaaaaagtggtcgctattc

SEQ ID NO: 84 - DNA, 38 bp, Artificial Sequence
  TRD-V-B-1
gtaaaacgacggccagttgcaaagaacctggctgtact

SEQ ID NO: 85 - DNA, 39 bp, Artificial Sequence
  TRD-V-C-1
gtaaaacgacggccagttgcagattttactcaaggacgg

SEQ ID NO: 86 - DNA, 38 bp, Artificial Sequence
  TRD-V-D-1
gtaaaacgacggccagtgcaaaatgcaacagaaggtcg

SEQ ID NO: 87 - DNA, 46 bp, Artificial Sequence
  TRD-V-E-1
gtaaaacgacggccagtgataaaaatgaagatggaagattcactgt

SEQ ID NO: 88 - DNA, 40 bp, Artificial Sequence
  TRD-V-F-1
gtaaaacgacggccagtctccttcaataaaagtgccaagc

SEQ ID NO: 89 - DNA, 44 bp, Artificial Sequence
  TRD-V-G-1
gtaaaacgacggccagtattgaaaagaagtcaggaagactaagt

SEQ ID NO: 90 - DNA, 37 bp, Artificial Sequence
  TRD-V-H-1
gtaaaacgacggccagttccagaaagcagccaaatcc

SEQ ID NO: 91 - DNA, 37 bp, Artificial Sequence
  TRD-D-A-1
gtaaaacgacggccagtaggggtattgtggatggcag

SEQ ID NO: 92 - DNA, 38 bp, Artificial Sequence
  TRD-J-A-1
taatacgactcactatagggttccacagtcacacgggt

SEQ ID NO: 93 - DNA, 41 bp, Artificial Sequence
  TRD-J-B-1
taatacgactcactatagggggttccacgatgagttgtgtt

SEQ ID NO: 94 - DNA, 41 bp, Artificial Sequence
  TRD-J-C-1
taatacgactcactatagggcacgaagagtttgatgccagt

SEQ ID NO: 95 - DNA, 43 bp, Artificial Sequence
  TRD-J-D-1
taatacgactcactataggggttgttgtacctccagataggtt

SEQ ID NO: 96 - DNA, 42 bp, Artificial Sequence
  TRD-J-E-1
taatacgactcactatagggtggctagaaacacttacttgca

SEQ ID NO: 97 - DNA, 41 bp, Artificial Sequence
  TRD-D-B-1
taatacgactcactatagggcccagggaaatggcacttttg

SEQ ID NO: 98 - DNA, 39 bp, Artificial Sequence
  IGH-D-A-1
gtaaaacgacggccagtgattcygaacagccccgagtca

SEQ ID NO: 99 - DNA, 38 bp, Artificial Sequence
  IGH-D-B-1
gtaaaacgacggccagtgattttgtgggggytcgtgtc

SEQ ID NO: 100 - DNA, 39 bp, Artificial Sequence
  IGH-D-C-1
gtaaaacgacggccagtgtttgrrgtgaggtctgtgtca

SEQ ID NO: 101 - DNA, 41 bp, Artificial Sequence
  IGH-D-D-1
gtaaaacgacggccagtgtttrgrrtgaggtctgtgtcact

SEQ ID NO: 102 - DNA, 39 bp, Artificial Sequence
  IGH-D-E-1
gtaaaacgacggccagtctttttgtgaaggsccctcctr

SEQ ID NO: 103 - DNA, 40 bp, Artificial Sequence
  IGH-D-F-1
gtaaaacgacggccagtgttattgtcaggsgrtgtcagac

SEQ ID NO: 104 - DNA, 40 bp, Artificial Sequence
  IGH-D-G-1
gtaaaacgacggccagtgttattgtcagggggtgycagrc

SEQ ID NO: 105 - DNA, 40 bp, Artificial Sequence
  IGH-D-H-1
gtaaaacgacggccagtgtttctgaagstgtctgtrtcac

SEQ ID NO: 106 - DNA, 42 bp, Artificial Sequence
  IGH-J-A-1
taatacgactcactatagggcttacctgaggagacggtgacc

SEQ ID NO: 107 - DNA, 40 bp, Artificial Sequence
  IGH-V-FR1-B-1
gtaaaacgacggccagtgcagtctggagcagaggtgaaaa

SEQ ID NO: 108 - DNA, 37 bp, Artificial Sequence
  IGH-V-FR1-E-1
gtaaaacgacggccagtgaggtgcagctgttggagtc

SEQ ID NO: 109 - DNA, 37 bp, Artificial Sequence
  IGH-V-FR1-G-1
gtaaaacgacggccagtcagtggggcgcaggactgtt

SEQ ID NO: 110 - DNA, 37 bp, Artificial Sequence
  IGH-V-FR1-H-1
gtaaaacgacggccagtccaggactggtgaagcctcc

SEQ ID NO: 111 - DNA, 41 bp, Artificial Sequence
  IGH-V-FR1-K-1
gtaaaacgacggccagtcctcagtgaaggtttcctgcaagg

SEQ ID NO: 112 - DNA, 43 bp, Artificial Sequence
  IGH-V-FR1-L-1
gtaaaacgacggccagtaaacccacagagaccctcacgctgac

SEQ ID NO: 113 - DNA, 42 bp, Artificial Sequence
  IGH-V-FR1-M-1
gtaaaacgacggccagtctggggggtccctgagactctcctg

SEQ ID NO: 114 - DNA, 42 bp, Artificial Sequence
  IGH-V-FR1-N-1
gtaaaacgacggccagtcttcacagaccctgtccctcacctg

SEQ ID NO: 115 - DNA, 42 bp, Artificial Sequence
  IGH-V-FR1-O-1
gtaaaacgacggccagttcgcagaccctctcactcacctgtg

SEQ ID NO: 116 - DNA, 42 bp, Artificial Sequence; identical to SEQ ID NO: 106
  IGH-J-A-1

SEQ ID NO: 117 - DNA, 42 bp, Artificial Sequence
  IGH-J-B-1
taatacgactcactatagggctcacctgaggagacggtgacc

SEQ ID NO: 118 - DNA, 37 bp, Artificial Sequence
  IGH-V-FR1-A-1
gtaaaacgacggccagtctggggctgaggtgaagaag

SEQ ID NO: 119 - DNA, 39 bp, Artificial Sequence
  IGH-V-FR1-C-1
gtaaaacgacggccagttcaccttgaaggagtctggtcc

SEQ ID NO: 120 - DNA, 36 bp, Artificial Sequence
  IGH-V-FR1-D-1
gtaaaacgacggccagtaggtgcagctggtggagtc

SEQ ID NO: 121 - DNA, 37 bp, Artificial Sequence
  IGH-V-FR1-F-1
gtaaaacgacggccagtccaggactggtgaagccttc

SEQ ID NO: 122 - DNA, 37 bp, Artificial Sequence
  IGH-V-FR1-I-1
gtaaaacgacggccagtgtacagctgcagcagtcagg

SEQ ID NO: 123 - DNA, 37 bp, Artificial Sequence
  IGH-V-FR1-J-1
gtaaaacgacggccagtgctggtgcaatctgggtctg

SEQ ID NO: 124 - DNA, 41 bp, Artificial Sequence
  IGK-V-A-1
gtaaaacgacggccagtaagtggggtcccatcaaggttcag

SEQ ID NO: 125 - DNA, 41 bp, Artificial Sequence
  IGK-V-B-1
gtaaaacgacggccagtagtcccatctcggttcagtggcag

SEQ ID NO: 126 - DNA, 41 bp, Artificial Sequence
  IGK-V-C-1
gtaaaacgacggccagtgaaacaggggtcccatcaaggttc

SEQ ID NO: 127 - DNA, 41 bp, Artificial Sequence
  IGK-V-D-1
gtaaaacgacggccagttcccagacagattcagtggcagtg

SEQ ID NO: 128 - DNA, 41 bp, Artificial Sequence
  IGK-V-E-1
gtaaaacgacggccagtctggagtgccagataggttcagtg

SEQ ID NO: 129 - DNA, 41 bp, Artificial Sequence
  IGK-V-F-1
gtaaaacgacggccagtccctggagtcccagacaggttcag

SEQ ID NO: 130 - DNA, 38 bp, Artificial Sequence
  IGK-V-G-1
gtaaaacgacggccagtgcatcccagccaggttcagtg

SEQ ID NO: 131 - DNA, 39 bp, Artificial Sequence
  IGK-V-H-1
gtaaaacgacggccagtgtccctgaccgattcagtggca

SEQ ID NO: 132 - DNA, 39 bp, Artificial Sequence
  IGK-V-I-1
gtaaaacgacggccagtaatcccacctcgattcagtggc

SEQ ID NO: 133 - DNA, 38 bp, Artificial Sequence
  IGK-V-J-1
gtaaaacgacggccagtctcaggggtcccctcgaggtt

SEQ ID NO: 134 - DNA, 37 bp, Artificial Sequence
  IGK-V-K-1
gtaaaacgacggccagtagacactggggtcccagcca

SEQ ID NO: 135 - DNA, 42 bp, Artificial Sequence
  IGK-DE-A-1
taatacgactcactataggggcagctgcagactcatgaggag

SEQ ID NO: 136 - DNA, 44 bp, Artificial Sequence
  IGk-J-A-1
taatacgactcactatagggacgtttgatctccaccttggtccc

SEQ ID NO: 137 - DNA, 44 bp, Artificial Sequence
  IGK-J-B-1
taatacgactcactatagggacgtttgatatccactttggtccc

SEQ ID NO: 138 - DNA, 44 bp, Artificial Sequence
  IGK-J-C-1
taatacgactcactatagggacgtttaatctccagtcgtgtccc

SEQ ID NO: 139 - DNA, 38 bp, Artificial Sequence
  IGK-INTR-A-1
gtaaaacgacggccagtgagtggctttggtggccatgc

SEQ ID NO: 140 - DNA, 42 bp, Artificial Sequence; identical to SEQ ID NO: 135
  IGK-DE-A-1

SEQ ID NO: 141 - DNA, 87 bp, Artificial Sequence
  Ill-D501-F
aatgatacggcgaccaccgagatctacactatagcctacactctttccctacacgacgctcttccgatctgtaaaacgacggccagt

SEQ ID NO: 142 - DNA, 87 bp, Artificial Sequence
  Ill-D502-F
aatgatacggcgaccaccgagatctacacatagaggcacactctttccctacacgacgctcttccgatctgtaaaacgacggccagt

SEQ ID NO: 143 - DNA, 87 bp, Artificial Sequence
  Ill-D503-F
aatgatacggcgaccaccgagatctacaccctatcctacactctttccctacacgacgctcttccgatctgtaaaacgacggccagt

SEQ ID NO: 144 - DNA, 87 bp, Artificial Sequence
  Ill-D504-F
aatgatacggcgaccaccgagatctacacggctctgaacactctttccctacacgacgctcttccgatctgtaaaacgacggccagt

SEQ ID NO: 145 - DNA, 87 bp, Artificial Sequence
  Ill-D505-F
aatgatacggcgaccaccgagatctacacaggcgaagacactctttccctacacgacgctcttccgatctgtaaaacgacggccagt

SEQ ID NO: 146 - DNA, 87 bp, Artificial Sequence
  Ill-D506-F
aatgatacggcgaccaccgagatctacactaatcttaacactctttccctacacgacgctcttccgatctgtaaaacgacggccagt

SEQ ID NO: 147 - DNA, 87 bp, Artificial Sequence
  Ill-D507-F
aatgatacggcgaccaccgagatctacaccaggacgtacactctttccctacacgacgctcttccgatctgtaaaacgacggccagt

SEQ ID NO: 148 - DNA, 87 bp, Artificial Sequence
  Ill-D508-F
aatgatacggcgaccaccgagatctacacgtactgacacactctttccctacacgacgctcttccgatctgtaaaacgacggccagt

SEQ ID NO: 149 - DNA, 86 bp, Artificial Sequence
  Ill-D701-R
caagcagaagacggcatacgagatcgagtaatgtgactggagttcagacgtgtgctcttccgatcttaatacgactcactataggg

SEQ ID NO: 150 - DNA, 86 bp, Artificial Sequence
  Ill-D702-R
caagcagaagacggcatacgagattctccggagtgactggagttcagacgtgtgctcttccgatcttaatacgactcactataggg

SEQ ID NO: 151 - DNA, 86 bp, Artificial Sequence
  Ill-D703-R
caagcagaagacggcatacgagataatgagcggtgactggagttcagacgtgtgctcttccgatcttaatacgactcactataggg

SEQ ID NO: 152 - DNA, 86 bp, Artificial Sequence
  Ill-D704-R
caagcagaagacggcatacgagatggaatctcgtgactggagttcagacgtgtgctcttccgatcttaatacgactcactataggg

SEQ ID NO: 153 - DNA, 86 bp, Artificial Sequence
  Ill-D705-R
caagcagaagacggcatacgagatttctgaatgtgactggagttcagacgtgtgctcttccgatcttaatacgactcactataggg

SEQ ID NO: 154 - DNA, 86 bp, Artificial Sequence
  Ill-D706-R
caagcagaagacggcatacgagatacgaattcgtgactggagttcagacgtgtgctcttccgatcttaatacgactcactataggg

SEQ ID NO: 155 - DNA, 86 bp, Artificial Sequence
  Ill-D707-R
caagcagaagacggcatacgagatagcttcaggtgactggagttcagacgtgtgctcttccgatcttaatacgactcactataggg

SEQ ID NO: 156 - DNA, 86 bp, Artificial Sequence
  Ill-D708-R
caagcagaagacggcatacgagatgcgcattagtgactggagttcagacgtgtgctcttccgatcttaatacgactcactataggg

SEQ ID NO: 157 - DNA, 86 bp, Artificial Sequence
  Ill-D709-R
caagcagaagacggcatacgagatcatagccggtgactggagttcagacgtgtgctcttccgatcttaatacgactcactataggg

SEQ ID NO: 158 - DNA, 86 bp, Artificial Sequence
  Ill-D710-R
caagcagaagacggcatacgagatttcgcggagtgactggagttcagacgtgtgctcttccgatcttaatacgactcactataggg

SEQ ID NO: 159 - DNA, 86 bp, Artificial Sequence
  Ill-D711-R
caagcagaagacggcatacgagatgcgcgagagtgactggagttcagacgtgtgctcttccgatcttaatacgactcactataggg

SEQ ID NO: 160 - DNA, 86 bp, Artificial Sequence
  Ill-D712-R
caagcagaagacggcatacgagatctatcgctgtgactggagttcagacgtgtgctcttccgatcttaatacgactcactataggg

SEQ ID NO: 161 - DNA, 21 bp, Artificial Sequence
  primer
tcgcttctcacctgaatgccc

SEQ ID NO: 162 - DNA, 22 bp, Artificial Sequence
  primer
ctcagttgaaaggcctgatgga

SEQ ID NO: 163 - DNA, 22 bp, Artificial Sequence
  primer
ggaagcatccctgatcgattct

SEQ ID NO: 164 - DNA, 22 bp, Artificial Sequence
  primer
tcagctaagtgcctcccaaatt

SEQ ID NO: 165 - DNA, 22 bp, Artificial Sequence
  primer
agttccaaatcgcttctcacct

SEQ ID NO: 166 - DNA, 22 bp, Artificial Sequence
  primer
ttccctaatcgattctcagggc

SEQ ID NO: 167 - DNA, 22 bp, Artificial Sequence
  primer
tacaactgccaaaggagaggtc

SEQ ID NO: 168 - DNA, 22 bp, Artificial Sequence
  primer
taaaggagaagtcccgaatggc

SEQ ID NO: 169 - DNA, 22 bp, Artificial Sequence
  primer
ggagaagttcccaatggctaca

SEQ ID NO: 170 - DNA, 22 bp, Artificial Sequence
  primer
ataaaggagaagtccccgatgg

SEQ ID NO: 171 - DNA, 22 bp, Artificial Sequence
  primer
ctctagatgattcggggatgcc

SEQ ID NO: 172 - DNA, 22 bp, Artificial Sequence
  primer
tgaagcagacacccctgataac

SEQ ID NO: 173 - DNA, 22 bp, Artificial Sequence
  primer
tgagcgatttttagcccaatgc

SEQ ID NO: 174 - DNA, 23 bp, Artificial Sequence
  primer
acaaaggagagatctctgatgga

SEQ ID NO: 175 - DNA, 22 bp, Artificial Sequence
  primer
ctacaactgtgagtctggtgcc

SEQ ID NO: 176 - DNA, 21 bp, Artificial Sequence
  primer
ctacaacggttaacctggtcc

SEQ ID NO: 177 - DNA, 22 bp, Artificial Sequence
  primer
tacaacagtgagccaacttccc

SEQ ID NO: 178 - DNA, 21 bp, Artificial Sequence
  primer
caagacagagagctgggttcc

SEQ ID NO: 179 - DNA, 22 bp, Artificial Sequence
  primer
ctaggatggagagtcgagtccc

SEQ ID NO: 180 - DNA, 20 bp, Artificial Sequence
  primer
ctgtcacagtgagcctggtc

SEQ ID NO: 181 - DNA, 22 bp, Artificial Sequence
  primer
ccttcttacctagcacggtgag

SEQ ID NO: 182 - DNA, 22 bp, Artificial Sequence
  primer
ttacccagtacggtcagcctag

SEQ ID NO: 183 - DNA, 20 bp, Artificial Sequence
  primer
cttaccgagcactgtcagcc

SEQ ID NO: 184 - DNA, 20 bp, Artificial Sequence
  primer
cttacccagcactgagagcc

SEQ ID NO: 185 - DNA, 19 bp, Artificial Sequence
  primer
tcaccgagcaccaggagcc

SEQ ID NO: 186 - DNA, 22 bp, Artificial Sequence
  primer
gaatctcacctgtgaccgtgag

SEQ ID NO: 187 - DNA, 21 bp, Artificial Sequence
  primer
ggaaacttccctggtcgattc

SEQ ID NO: 188 - DNA, 22 bp, Artificial Sequence
  primer
caacgatcggttctttgcagtc

SEQ ID NO: 189 - DNA, 22 bp, Artificial Sequence
  primer
taaatcagggctgctcagtgat

SEQ ID NO: 190 - DNA, 22 bp, Artificial Sequence
  primer
cagtgatcggttctctgcagag

SEQ ID NO: 191 - DNA, 22 bp, Artificial Sequence
  primer
cttgaacgattctccgcacaac

SEQ ID NO: 192 - DNA, 22 bp, Artificial Sequence
  primer
ccgaggatcgattctcagctaa

SEQ ID NO: 193 - DNA, 22 bp, Artificial Sequence
  primer
gccaaaggaacgattttctgct

SEQ ID NO: 194 - DNA, 22 bp, Artificial Sequence
  primer
agggagatgttcctgaagggta

SEQ ID NO: 195 - DNA, 22 bp, Artificial Sequence
  primer
cctgaggggtacagtgtctcta

SEQ ID NO: 196 - DNA, 22 bp, Artificial Sequence
  primer
cagaatctctcagcctccagac

SEQ ID NO: 197 - DNA, 22 bp, Artificial Sequence
  primer
acttccctgatcgattctcagc

SEQ ID NO: 198 - DNA, 22 bp, Artificial Sequence
  primer
ctcaggtcaccagttccctaac

SEQ ID NO: 199 - DNA, 22 bp, Artificial Sequence
  primer
cctagattttcaggtcgccagt

SEQ ID NO: 200 - DNA, 22 bp, Artificial Sequence
  primer
ctcaactagacaaatcggggct

SEQ ID NO: 201 - DNA, 22 bp, Artificial Sequence
  primer
atcgattttctgcagagaggct

SEQ ID NO: 202 - DNA, 22 bp, Artificial Sequence
  primer
cggtatgcccaacaatcgattc

SEQ ID NO: 203 - DNA, 21 bp, Artificial Sequence
  primer
ctgaagggtacagcgtctctc

SEQ ID NO: 204 - DNA, 22 bp, Artificial Sequence
  primer
tcctctgagtcaacagtctcca

SEQ ID NO: 205 - DNA, 22 bp, Artificial Sequence
  primer
ctgaggccacatatgagagtgg

SEQ ID NO: 206 - DNA, 21 bp, Artificial Sequence
  primer
gaaaactcacccagcacggtc

SEQ ID NO: 207 - DNA, 19 bp, Artificial Sequence
  primer
tcacccagcacggtcagcc

SEQ ID NO: 208 - DNA, 22 bp, Artificial Sequence
  primer
gattctcaggtctccagttccc

SEQ ID NO: 209 - DNA, 22 bp, Artificial Sequence
  primer
taccactggcaaaggagaagtc

SEQ ID NO: 210 - DNA, 22 bp, Artificial Sequence
  primer
caaaggagaagtctcagatggc

SEQ ID NO: 211 - DNA, 22 bp, Artificial Sequence
  primer
tttctcatcaaccatgcaagcc

SEQ ID NO: 212 - DNA, 22 bp, Artificial Sequence
  primer
ggagatgcacaagaagcgattc

SEQ ID NO: 213 - DNA, 22 bp, Artificial Sequence
  primer
ctacaactgtgagtctggtgcc

SEQ ID NO: 214 - DNA, 21 bp, Artificial Sequence
  primer
ctacaacggttaacctggtcc

SEQ ID NO: 215 - DNA, 22 bp, Artificial Sequence
  primer
tacaacagtgagccaacttccc

SEQ ID NO: 216 - DNA, 21 bp, Artificial Sequence
  primer
caagacagagagctgggttcc

SEQ ID NO: 217 - DNA, 22 bp, Artificial Sequence
  primer
ctaggatggagagtcgagtccc

SEQ ID NO: 218 - DNA, 20 bp, Artificial Sequence
  primer
ctgtcacagtgagcctggtc

SEQ ID NO: 219 - DNA, 22 bp, Artificial Sequence
  primer
ccttcttacctagcacggtgag

SEQ ID NO: 220 - DNA, 22 bp, Artificial Sequence
  primer
ttacccagtacggtcagcctag

SEQ ID NO: 221 - DNA, 20 bp, Artificial Sequence
  primer
cttaccgagcactgtcagcc

SEQ ID NO: 222 - DNA, 20 bp, Artificial Sequence
  primer
cttacccagcactgagagcc

SEQ ID NO: 223 - DNA, 19 bp, Artificial Sequence
  primer
tcaccgagcaccaggagcc

SEQ ID NO: 224 - DNA, 22 bp, Artificial Sequence
  primer
gaatctcacctgtgaccgtgag

SEQ ID NO: 225 - DNA, 20 bp, Artificial Sequence
  primer
cctccactcccctcaaagga

SEQ ID NO: 226 - DNA, 21 bp, Artificial Sequence
  primer
cagactaacctctgccacctg

SEQ ID NO: 227 - DNA, 21 bp, Artificial Sequence
  primer
gaaaactcacccagcacggtc

SEQ ID NO: 228 - DNA, 19 bp, Artificial Sequence
  primer
tcacccagcacggtcagcc

SEQ ID NO: 229 - DNA, 26 bp, Artificial Sequence
  primer
caagcatgaggaggagctggaaattg

SEQ ID NO: 230 - DNA, 21 bp, Artificial Sequence
  primer
acgtctacatccactctcacc

SEQ ID NO: 231 - DNA, 23 bp, Artificial Sequence
  primer
gcacaaggaacaacttgagattg

SEQ ID NO: 232 - DNA, 26 bp, Artificial Sequence
  primer
tggaagcacaaggaagaacttgagaa

SEQ ID NO: 233 - DNA, 22 bp, Artificial Sequence
  primer
gcacagggaagagccttaaatt

SEQ ID NO: 234 - DNA, 21 bp, Artificial Sequence
  primer
caggaggtggagctggatatt

SEQ ID NO: 235 - DNA, 25 bp, Artificial Sequence
  primer
ctctcacttcaatccttaccatcaa

SEQ ID NO: 236 - DNA, 34 bp, Artificial Sequence
  primer
gctcacacttccacttccactttgaaaataaagt

SEQ ID NO: 237 - DNA, 21 bp, Artificial Sequence
  primer
agtgttgttccactgccaaag

SEQ ID NO: 238 - DNA, 21 bp, Artificial Sequence
  primer
gttccgggaccaaataccttg

SEQ ID NO: 239 - DNA, 23 bp, Artificial Sequence
  primer
gagcttagtcccttcagcaaata

SEQ ID NO: 240 - DNA, 20 bp, Artificial Sequence
  primer
cctagtcccttttgcaaacg

SEQ ID NO: 241 - DNA, 24 bp, Artificial Sequence
  primer
gaatgcaaaaagtggtcgctattc

SEQ ID NO: 242 - DNA, 21 bp, Artificial Sequence
  primer
tgcaaagaacctggctgtact

SEQ ID NO: 243 - DNA, 22 bp, Artificial Sequence
  primer
tgcagattttactcaaggacgg

SEQ ID NO: 244 - DNA, 21 bp, Artificial Sequence
  primer
gcaaaatgcaacagaaggtcg

SEQ ID NO: 245 - DNA, 29 bp, Artificial Sequence
  primer
gataaaaatgaagatggaagattcactgt

SEQ ID NO: 246 - DNA, 23 bp, Artificial Sequence
  primer
ctccttcaataaaagtgccaagc

SEQ ID NO: 247 - DNA, 27 bp, Artificial Sequence
  primer
attgaaaagaagtcaggaagactaagt

SEQ ID NO: 248 - DNA, 20 bp, Artificial Sequence
  primer
tccagaaagcagccaaatcc

SEQ ID NO: 249 - DNA, 20 bp, Artificial Sequence
  primer
aggggtattgtggatggcag

SEQ ID NO: 250 - DNA, 18 bp, Artificial Sequence
  primer
ttccacagtcacacgggt

SEQ ID NO: 251 - DNA, 21 bp, Artificial Sequence
  primer
ggttccacgatgagttgtgtt

SEQ ID NO: 252 - DNA, 21 bp, Artificial Sequence
  primer
cacgaagagtttgatgccagt

SEQ ID NO: 253 - DNA, 23 bp, Artificial Sequence
  primer
gttgttgtacctccagataggtt

SEQ ID NO: 254 - DNA, 22 bp, Artificial Sequence
  primer
tggctagaaacacttacttgca

SEQ ID NO: 255 - DNA, 21 bp, Artificial Sequence
  primer
cccagggaaatggcacttttg

SEQ ID NO: 256 - DNA, 22 bp, Artificial Sequence
  primer
gattcygaacagccccgagtca

SEQ ID NO: 257 - DNA, 21 bp, Artificial Sequence
  primer
gattttgtgggggytcgtgtc

SEQ ID NO: 258 - DNA, 22 bp, Artificial Sequence
  primer
gtttgrrgtgaggtctgtgtca

SEQ ID NO: 259 - DNA, 24 bp, Artificial Sequence
  primer
gtttrgrrtgaggtctgtgtcact

SEQ ID NO: 260 - DNA, 22 bp, Artificial Sequence
  primer
ctttttgtgaaggsccctcctr

SEQ ID NO: 261 - DNA, 23 bp, Artificial Sequence
  primer
gttattgtcaggsgrtgtcagac

SEQ ID NO: 262 - DNA, 23 bp, Artificial Sequence
  primer
gttattgtcagggggtgycagrc

SEQ ID NO: 263 - DNA, 23 bp, Artificial Sequence
  primer
gtttctgaagstgtctgtrtcac

SEQ ID NO: 264 - DNA, 22 bp, Artificial Sequence
  primer
cttacctgaggagacggtgacc

SEQ ID NO: 265 - DNA, 23 bp, Artificial Sequence
  primer
gcagtctggagcagaggtgaaaa

SEQ ID NO: 266 - DNA, 20 bp, Artificial Sequence
  primer
gaggtgcagctgttggagtc

SEQ ID NO: 267 - DNA, 20 bp, Artificial Sequence
  primer
cagtggggcgcaggactgtt

SEQ ID NO: 268 - DNA, 20 bp, Artificial Sequence
  primer
ccaggactggtgaagcctcc

SEQ ID NO: 269 - DNA, 24 bp, Artificial Sequence
  primer
cctcagtgaaggtttcctgcaagg

SEQ ID NO: 270 - DNA, 26 bp, Artificial Sequence
  primer
aaacccacagagaccctcacgctgac

SEQ ID NO: 271 - DNA, 25 bp, Artificial Sequence
  primer
ctggggggtccctgagactctcctg

SEQ ID NO: 272 - DNA, 25 bp, Artificial Sequence
  primer
cttcacagaccctgtccctcacctg

SEQ ID NO: 273 - DNA, 25 bp, Artificial Sequence
  primer
tcgcagaccctctcactcacctgtg

SEQ ID NO: 274 - DNA, 22 bp, Artificial Sequence
  primer
cttacctgaggagacggtgacc

SEQ ID NO: 275 - DNA, 22 bp, Artificial Sequence
  primer
ctcacctgaggagacggtgacc

SEQ ID NO: 276 - DNA, 20 bp, Artificial Sequence
  primer
ctggggctgaggtgaagaag

SEQ ID NO: 277 - DNA, 22 bp, Artificial Sequence
  primer
tcaccttgaaggagtctggtcc

SEQ ID NO: 278 - DNA, 19 bp, Artificial Sequence
  primer
aggtgcagctggtggagtc

SEQ ID NO: 279 - DNA, 20 bp, Artificial Sequence
  primer
ccaggactggtgaagccttc

SEQ ID NO: 280 - DNA, 20 bp, Artificial Sequence
  primer
gtacagctgcagcagtcagg

SEQ ID NO: 281 - DNA, 20 bp, Artificial Sequence
  primer
gctggtgcaatctgggtctg

SEQ ID NO: 282 - DNA, 24 bp, Artificial Sequence
  primer
aagtggggtcccatcaaggttcag

SEQ ID NO: 283 - DNA, 24 bp, Artificial Sequence
  primer
agtcccatctcggttcagtggcag

SEQ ID NO: 284 - DNA, 24 bp, Artificial Sequence
  primer
gaaacaggggtcccatcaaggttc

SEQ ID NO: 285 - DNA, 24 bp, Artificial Sequence
  primer
tcccagacagattcagtggcagtg

SEQ ID NO: 286 - DNA, 24 bp, Artificial Sequence
  primer
ctggagtgccagataggttcagtg

SEQ ID NO: 287 - DNA, 24 bp, Artificial Sequence
  primer
ccctggagtcccagacaggttcag

SEQ ID NO: 288 - DNA, 21 bp, Artificial Sequence
  primer
gcatcccagccaggttcagtg

SEQ ID NO: 289 - DNA, 22 bp, Artificial Sequence
  primer
gtccctgaccgattcagtggca

SEQ ID NO: 290 - DNA, 22 bp, Artificial Sequence
  primer
aatcccacctcgattcagtggc

SEQ ID NO: 291 - DNA, 21 bp, Artificial Sequence
  primer
ctcaggggtcccctcgaggtt

SEQ ID NO: 292 - DNA, 20 bp, Artificial Sequence
  primer
agacactggggtcccagcca

SEQ ID NO: 293 - DNA, 22 bp, Artificial Sequence
  primer
gcagctgcagactcatgaggag

SEQ ID NO: 294 - DNA, 24 bp, Artificial Sequence
  primer
acgtttgatctccaccttggtccc

SEQ ID NO: 295 - DNA, 24 bp, Artificial Sequence
  primer
acgtttgatatccactttggtccc

SEQ ID NO: 296 - DNA, 24 bp, Artificial Sequence
  primer
acgtttaatctccagtcgtgtccc

SEQ ID NO: 297 - DNA, 21 bp, Artificial Sequence
  primer
gagtggctttggtggccatgc

SEQ ID NO: 298 - DNA, 22 bp, Artificial Sequence
  primer
gcagctgcagactcatgaggag

(19) United States
(12) Patent Application Publication
(10) Pub. No.: US 2024/0018596 A1
(43) Pub. Date: Jan. 18, 2024

(54) MEANS AND METHODS FOR ACCURATELY ASSESSING CLONAL IMMUNOGLOBULIN (IG)/T CELL RECEPTOR (TR) GENE REARRANGEMENTS

(71) Applicant: Stichting EuroClonality, Zutphen (NL)

(72) Inventors: Monika Ursula Helga Brüggemann, Marsberg (DE); Michaela Kotrová, Frydek-Mistek (CZ); Henrik Knecht, Neustrelitz (DE); Nikolaos Darzentas, Athens (GR); Giovanni Cazzaniga, Vimercate (IT); Frédéric Bernard Louis Davi, Paris (FR); Jacobus Johannes Maria Van Dongen, Bergen op Zoom (NL); Ramón Garcia-Sanz, Salamanca (ES); David Gonzalez De Castro, Madrid (ES); Patricia Johanna Theodore Groenen, Elst (NL); Michael Andreas Hummel, Ellwangen (DE); Elizabeth Anne Macintyre-Davi, Inverness (GB); Konstantinos Stamatopoulos, Athens (GR); Christiane Pott, Leverkusen (DE); Jan Trka, Prague (CZ); Anthonie Willem Langerak, Rotterdam (NL)

(21) Appl. No.: 17/440,010

(22) PCT Filed: Mar. 18, 2020
(86) PCT No.: PCT/NL2020/050181

§ 371 Date: Sep. 16, 2021

(30) Foreign Application Priority Data
Mar. 19, 2019 (EP) 19163837.8

(51) Int. Cl. C12Q 1/6886; C12Q 1/6879

(52) U.S. Cl. CPC C12Q 1/6886; C12Q 1/6879; C12Q 2600/156; C12Q 2600/16; C12Q 2600/166; C12Q 1/6869

(57) ABSTRACT

The invention relates to means and methods for assessing clonal immunoglobulin (IG)/T cell receptor (TR) gene rearrangements in a clinical, diagnostic and/or research setting. Provided is a quality control composition comprising a mixture of genomic DNA isolated from a set of nine cultured cell lines, said set comprising the B cell lines ALL/MIK (ALL), Raji (Burkitt lymphoma), REH (B cell precursor ALL), TMM (CML-BC/EBV+B-LCL), TOM-1 (B cell precursor ALL), WSU-NHL (B cell lymphoma) and the T cell lines JB6 (ALCL), Karpas299 (ALCL) and MOLT-13 (ALL), or wherein one or more cell lines of said set is replaced with one or more other cell line(s) comprising the same IG/TR gene rearrangements. Also provided is a quality control composition consisting of essentially equimolar amounts of genomic DNA isolated from healthy human thymus, healthy human tonsil and healthy human peripheral blood mononuclear cells.

### INCORPORATION OF SEQUENCE LISTING

The file named “VONL022US-corrected-8-18-2022.txt” contains a computer readable form of the Sequence Listing and was created on Aug. 18, 2022. The file is 56,268 bytes (measured in MS-Windows®), is filed contemporaneously along with this application by electronic submission (using the United States Patent Office EFS-Web filing system), and is incorporated herein by reference in its entirety.

The invention relates to the fields of immunology, immunogenetics and clinical diagnostics. In particular, it relates to means and methods for assessing clonal immunoglobulin (IG)/T cell receptor (TR) gene rearrangements in a clinical, diagnostic and/or research setting.

Specific antigen recognition by cells of the adaptive immune system (B cells, T cells) is mediated through receptors (immunoglobulin, IG, and T cell receptor, TR) that are uniquely formed during immune development in bone marrow and thymus, respectively. Through recombination of IG/TR loci a diverse (polyclonal) repertoire of unique IG/TR receptors is created. In certain autoimmune diseases this repertoire is skewed (oligoclonal), whereas in lymphoid malignancies receptors are largely identical (monoclonal)¹⁻⁷. IG/TR rearrangements thus form unique genetic biomarkers (molecular signatures) for studying immune cells for clinical, diagnostic and research applications⁸⁻¹¹. Classically, methods for immunogenetic analysis mostly concern fragment analysis and Sanger-based sequencing. The introduction of next-generation sequencing (NGS) makes deeper analysis of IG/TR rearrangements possible, with impact on the main immunogenetic applications: clonality assessment, minimal residual disease (MRD) detection, repertoire analysis¹²⁻²⁹.

Identification and assessment of clonal IG/TR gene rearrangements is a widely used tool for the diagnosis and follow-up of lymphoid malignancies³⁰⁻³⁵. NGS of IG/TR gene rearrangements is gaining popularity in clinical laboratories, as it avoids laborious design of patient-specific real-time quantitative (RQ)-PCR assays and provides the capability to sequence multiple rearrangements and rearrangement types within a single sequencing run. Hence, several methods have already been described for high-throughput profiling of IG/TR rearrangements at diagnosis and follow-up in acute lymphoblastic leukemia (ALL), chronic lymphocytic leukemia (CLL) and other lymphoid malignancies.^{16,17,23,24,36,37}

Potential applications for IG/TR NGS are identification of clonal IG/TR markers in diagnostic samples for subsequent analysis of minimal residual disease (MRD), but also the actual MRD analysis itself, in different lymphoid malignancies (mainly ALL, CLL, follicular lymphoma, mantle cell lymphoma, multiple myeloma). In addition, it can be applied for clonality diagnostics in the diagnostic process of lymphoproliferative disorders.

It has been found that NGS assays, especially those based on amplicons, pose major challenges. For example, multiple primers need to anneal under the same reaction conditions, while many technical variables may be introduced by sample preparation, library construction, sequencing and bioinformatics, potentially leading to inaccurate results³⁸. Unfortunately, standardization and validation in a scientifically-controlled multicentre setting is still lacking. Particularly in a clinical context, strategies for standardisation of laboratory protocols and quality control (QC) of each component of an NGS assay are highly sought for.

Reference standards are essential for the evaluation of wet-lab and in silico NGS processes to ensure the analytical validity of test results prior to implementation of an NGS technology into clinical practice^{29,39,40}. Reference DNA materials should be stable sources of rearrangements that can be sequenced and used for measuring qualitative and quantitative properties. However, the present inventors recognized that previously published standards have a limited scope and utility, since they (1) do not cover all relevant IG/TR loci, (2) do not report on the quality of the sequencing run or the performance of samples and primers, and/or (3) are synthetic constructs that may not reflect the complexity of native genomic DNA^{23,41,42}.

Therefore, they aimed at providing improved types of quality controls that can be readily integrated in existing systems for immunoprofiling IG/TR sequence data, in particular ARResT/Interrogate⁴³, an interactive web-based computational platform that can process and annotate large amounts of immunogenetic data, calculate several relevant statistics including on QC, and present results in the form of multiple interconnected visualizations. More specifically, they sought to provide a composition that can be directly added to a sample to undergo concurrent library preparation and sequencing, acting as in-tube qualitative and quantitative standard that is subjected to the same technical downstream variables as the accompanying samples. Furthermore, they aimed at providing a composition that allows to uniformly assess performance biases or unusual amplification shifts across all types of IG/TR gene rearrangements in a sequencing run by tracking primer usage and comparison with stored reference profiles.

To that end, the present inventors of the EuroClonality-NGS Working Group joined forces to develop, standardize and validate in vitro assays and bioinformatics for IG/TR NGS applications. This resulted in the provision of two novel types of quality controls; a central in-tube quality/quantification control (cIT-QC) based on human B and T cell lines with well-defined IG/TR gene rearrangements, and a central polytarget quality control (cPT-QC) based on a standardised mixture of lymphoid specimens representing a full repertoire of IG/TR genes.

Accordingly, in a first aspect the invention provides a composition (herein also referred to as “central in-tube quality/quantification control” (cIT-QC)) comprising a mixture of genomic DNA isolated from a set of nine cultured cell lines, said set comprising the B cell lines ALL/MIK (B cell precursor ALL), Raji (Burkitt lymphoma), REH (B cell precursor ALL), TMM (CML-BC/EBV+B-LCL), TOM-1 (B cell precursor ALL), WSU-NHL (B cell lymphoma); and three T cell lines: JB6 (ALCL), Karpas299 (ALCL) and MOLT-13 (T-ALL), or wherein one or more cell lines of said set is replaced with one or more other cell line(s) comprising the same immunoglobulin (IG)/T cell receptor (TR) gene rearrangements, i.e. the same IG/TR rearrangement profile.

In a second aspect, the invention provides a composition (herein also referred to as “central polytarget quality control” (cPT-QC)) consisting of essentially equimolar amounts of genomic DNA isolated from healthy human thymus, healthy human tonsil and healthy human peripheral blood mononuclear cells.

Compositions according to the present invention are not known or suggested in the art. US2018/208984 A1 relates to a method for detecting IG/TR rearrangements using next-generation sequencing using a set of primers. A set of plasmids comprising known alleles, including TCR sequences of the T cell lines JB6, Karpas299 and MOLT-13, is used as a control. However, the control sample of US2018/208984 using cDNA prevents the inclusion of incomplete TR and IG rearrangements, because they are not transcribed into mRNA molecules. Such incomplete TR and IG rearrangements are explicit targets in the present invention, as they are complementary targets for clonality detection and MRD assessment. For example, unlike a control composition of the invention, US2018/208984 does not allow for the identification and quantification of the rearrangements TRB D-J, TRD V-D, TRD D-D, TRD D-J, IGK-Kde and IGH D-J.

Beccutti et al. (BMC Bioinformatics, Vol. 18, no. 1, 2017, pages 1-12) relates to a method for detecting IG/TR rearrangements using NGS. DNA isolated from buffy coat (comprising peripheral blood mononuclear cells) is used as a control. Beccutti et al. is silent about the use of DNA from additional tissue sources, let alone that it suggests to include tonsil and thymus genomic DNA in a polytarget quality control composition in order to include essential rearrangements that are not found in PBMCs.

A cIT-QC composition as provided herein has a number of unique and advantageous properties. First, with the selected set of only nine cell lines featuring a total of 46 rearrangements, it represents as few cell lines as possible, while covering each target by at least three different rearrangements, hence allowing for detecting ALL cells harbouring not only lineage-associated but also cross-lineage rearrangements. Second, the rearrangements are unambiguously detectable with Sanger sequencing and/or amplicon-based NGS. Third, the variable region of IGH gene rearrangements are unmutated and therewith avoid issues with primer annealing. Table 1 presents the full list of the 46 rearrangements.

With the use of genomic DNA, a composition of the invention explicitly avoids the usage of plasmids, which are known to pose a serious threat to contaminate PCR assays. Additionally, genomic DNA was chosen to optimally represent the patient samples for which the assay is intended for, and which also comprise genomic DNA.

Genomic DNA is readily isolated from the cell lines using established extraction protocols known in the art. In one embodiment, the DNA is obtained using a phenol-chloroform extraction protocol, followed by ethanol precipitation and elution in Tris ethylenediaminetetra-acetic acid (TE) buffer. The composition is suitably in a dry (e.g. lyophilized) form to be reconstituted with a liquid prior to use.

TABLE 1
Composition and IG/TR rearrangement profile
of cell lines used in the cIT-QC composition.

| Lineage | Entity | Cell line | IGH-DJ | IGH-VJ | IGK-VJ-Kde |
|---|---|---|---|---|---|
| T | ALCL | JB6 | | | |
| T | ALCL | Karpas299 | | | |
| T | T-ALL | MOLT-13 | | | |
| B | B cell | ALL/MIK | | V3-72 | V1D-39 = V1-39 |
| | precursor | | | 16/24/J4; | 6/7/5 J3; |
| | ALL | | | V7-4-1 | V2D-24 = V2-24 |
| | | | | 11/40/27 J4 | 26/6/20 Kde |
| B | B cell | REH | | V3-15 | V2-29 5/4/J4; |
| | precursor | | | 1/21/5 J6 | V3D-20 = V3-20 |
| | ALL | | | | 4/1/Kde |
| B | Burkitt | Raji | D6-13 | V3-11 = V3- | V1-8 2/2/4 Kde |
| | Lymphoma | | 8/12/6 J1 | 21 = V3-48 | |
| | | | | 2/40/3 J4 | |
| B | CML-BC/ | TMM | D2-2 | V1-24/28/8 | V2D-30 = V2- |
| | EBV + B − | | 3/13/J3 | J5 | 30/7/3 Kde |
| | LCL | | | | |
| B | B cell | TOM-1 | | V4-55 | |
| | precursor | | | 1/17/10 J6 | |
| | ALL | | | | |
| B | B cell | WSU-NHL | D2-2 | V6-1 | V1D-17 = V1-17 |
| | lymphoma | | 1/1/8 J4 | 1/22/19 J6 | 1//4 J4 |

| Lineage | intron-Kde | TRB-DJ | TRB-VJ | TRD | TRG |
|---|---|---|---|---|---|
| T | | D1 7/6/4 J2-2 | V12-3 = V12-4 | | V10 7/12/12 |
| | | | 6/14/4 J2-3 | | J1 = J2; |
| | | | | | V2 5/13/J1 = J2 |
| T | | D1/2/6 J1-6 | V20-1 1/22/6 | | V2/13/4 JP2; |
| | | | J2-7 | | V8/2/5 J1 = J2 |
| T | | D1//6 J1-5; | V10-1 6/18/1 | V1 1/9/J1 | V3/8/9 J1 = J2; |
| | | D2/4/3 J2-3 | J1-1 | | V8 3//3 JP1 |
| B | intron | | | V2 5/21/4 | V2/5/8 JP1; |
| | 4/2/Kde; | | | J29 | V5 2/3/JP1 |
| | intron | | | | |
| | 4/6/1 Kde | | | | |
| B | intron | | V20-1 1/2/26 | V2 3/22/5 | V4 10/14/3 |
| | 5//Kde | | J2-7 | J29; | J1 = J2; |
| | | | | V2 7/3/D3 | V9 1/2/3 J1 = J2 |
| B | | | | | |
| B | | | | | |
| B | | | | V2 3/3/2 D3; | V5 8//18 J1 = J2 |
| | | | | V2 8/4/D3 | |
| B | intron | | | | |
| | 2//3 Kde | | | | |

In a preferred aspect, a composition comprises a mixture of about equal amounts of genomic DNA isolated from the selected set of cultured cell lines. For example, the cIT-QC composition is formulated to provide a test sample with the DNA of at least 40 cell copies, preferably at least 50 copies of each cell line. Whereas there is no maximum number of cell copies to be represented in the control sample, very high amounts of genomic DNA may consume to a considerable extent the sequencing power in the assay. In one embodiment, the cIT-QC composition contains about an equal number of cell line DNA copies of the selected set of cultured cell lines and is (formulated to be) reconstituted to a solution that contains the genomic DNA of 20-50 cell copies of each cell line per reaction.

The B and T cell lines for use in a cIT-QC composition provided herein can be obtained from any suitable (commercial) source. For example, the Raji cell line is DSMZ ACC 319, the REH cell line is DSMZ ACC 22, the TMM cell line is DSMZ ACC 95, the TOM-1 cell line is DSMZ ACC 578, the WSU-NHL cell line is DSMZ ACC 58, the Karpas299 cell line is DSMZ ACC 31 and/or the MOLT-13 cell line is DSMZ ACC 436. It is of course also possible to replace one or more of the cell lines shown in Table 1 with another cell line having good growth characteristics that contains (or is provided with) the same rearrangements. Hence, also encompassed is a composition comprising a mixture of genomic DNA isolated from a set of cultured cell lines which together cover the profile with 46 rearrangement types shown in Table 1. In particular, the invention provides a composition comprising a mixture of genomic DNA isolated from a set of nine cultured cell lines, said set comprising the B cell lines ALL/MIK (B cell precursor ALL), Raji (Burkitt lymphoma), REH (B cell precursor ALL), TMM (CML-BC/EBV+B-LCL), TOM-1 (B cell precursor ALL), WSU-NHL (B cell lymphoma) and the T cell lines JB6 (ALCL), Karpas299 (ALCL) and MOLT-13 (T-ALL), or wherein one or more cell lines of said set is replaced with one or more other (i.e. distinct from the nine cell lines recited), cell line(s) comprising the same immunoglobulin (IG)/T cell receptor (TR) gene rearrangements. Such an “equivalent” cell type comprises at least the same gene rearrangements depicted in Table 1, or IG/TR rearrangements of the same type, i.e. different CDR3. In a preferred aspect, the composition comprises genomic DNA isolated from the B cell lines ALL/MIK, REH and TOM-1, each comprising cross-lineage TR rearrangements.

In a preferred embodiment, the composition consists of a mixture of, preferably in about equal amounts, genomic DNA isolated from the B cell lines ALL/MIK, Raji, REH, TMM, TOM-1, WSU-NHL and the T cell lines JB6, Karpas299 and MOLT-13.

In a further aspect, the invention provides a cPT-QC composition consisting of essentially equimolar amounts of genomic DNA isolated from healthy human thymus, healthy human tonsil and healthy human peripheral blood mononuclear cells (PB-MNC). In other words, it consists of an equimolar mixture of ⅓ thymus, ⅓ tonsil, ⅓ PB-MNC DNA. As used herein, the term “healthy” refers to tissue obtained from a human subject that is known or presumed not to suffer from an underlying malignant immunological disease or disorder. In one aspect, thymus is obtained from young children through removal due to physical impossibility to reach the heart for surgery. It is preferred that, for each tissue, the genomic DNA is obtained from a number of different human individuals. For example, for each tissue the DNA of 3 to 10 human subjects is used. Since this composition represents a “standardised lymphoid specimen”, it is suitably used as separate sample to be processed alongside test samples, it is preferably formulated to provide essentially the same amount of DNA as a regular sample that is tested. This typically ranges from 50 to 200 ng, preferably. In a specific aspect, the composition is dried e.g. lyophilized.

The expression “in about equal amounts” or “in essentially equal amounts” as used herein reflects the aim that each of the cell lines/lymphoid tissue samples is equally represented in the mixture of genomic DNA.

The invention also provides a diagnostic kit comprising a (first) container comprising a “central in-tube quality/quantification control” (cIT-QC) composition of the invention and/or a (second) container comprising a “central polytarget quality control” (cPT-QC) composition as herein disclosed. In one embodiment, the kit comprises at least a cIT-QC composition as herein disclosed. In another embodiment, the kit comprises at least a cPT-QC composition of the present invention. The cPT-QC composition may be packaged together with one or more further useful quality control composition(s). For example, the further control composition may comprise a mixture of genomic DNA isolated from a set of cultured cell lines which together cover the profile with 46 rearrangement types shown in Table 1, such that both quality controls can be used to monitor the assay performance when assessing clonal IG/TR gene rearrangements.

Preferably, the kit comprises both the cIT-QC and the cPT-QC compositions as herein disclosed. The kit may advantageously further comprise one or more reagents for detecting IG/TR gene rearrangements, such as a set of primers for amplicon-based NGS of IG/TR gene rearrangements. In a specific embodiment, the diagnostic kit comprises, in addition to one or both QC composition(s) provided herein, one or more primer sets for detecting one or more of the IG/TR gene rearrangements selected from the group consisting of IGH-VJ, IGH-DJ, IGK-VJ-Kde, TRB-VJ, TRB-DJ, TRD and TRG. Particularly preferred primers, e.g. for use in combination with the QC compositions, are those that have been optimized for NGS-based detection, such as the primers shown in FIG. 5 or Table 3.

The invention also provides a set of primers for amplicon-based next-generation sequencing (NGS) of IG/TR gene rearrangements, comprising two or more of the primers selected from the primers shown in FIG. 5. Preferably, the set comprises at least two primers for detecting one or more, preferably two or more, more preferably three or more, of the IG/TR gene rearrangements selected from the group consisting of IGH-VJ, IGH-DJ, IGK-VJ-Kde, TRB-VJ, TRB-DJ, TRD and TRG. In a specific aspect, the primer set comprises primers for detecting each of the IGH-VJ, IGH-DJ, IGK-VJ-Kde, TRB-VJ, TRB-DJ, TRD and TRG gene rearrangements. The primer sequences of FIG. 5 may be provided with a universal primer sequences (such as M13 forward sequence M13 forward primer (−20): GTAAAACGACGGCCAGT; and/or T7 universal primer: TAATACGACTCACTATAGGG) or other universal primer sequences known in the art that do not hybridize to the target sequence, and/or other adaptor or bar code sequences.

In a specific aspect, a primer of the invention comprises a forward or reverse M13 sequence. In one embodiment, a primer sequence of FIG. 5 is provided with a universal M13 tail at its 5⋅end, preferably with the sequence GTAAAACGACGGCCAGT. In another embodiment, a primer sequence of FIG. 5 is provided with a T7 promotor sequence. In a specific aspect, the invention provide a set of primers comprises two or more of the primers selected from the primers shown in Table 3.

The provision of the novel QC compositions of the invention has important implications for the quality control and quantitation strategies. As is demonstrated herein below, the cPT-QC composition is a valuable tool to monitor reproducibility of results and to identify primer perturbations and other deviations in the wet lab protocol, as they introduce detectable changes to the sequencing profile. The addition of the cPT-QC to each sequencing run allows to check the primer and assay performance after sequencing. Accidental deviations in the concentrations of single primers within the multiplexed IG/TR primer sets can be detected, performance failures of single primers can be traced and consequences for the IG/TR analysis can be estimated by analysis of the cPT-QC data.

Additionally, the advantages and diverse utility of the cIT-QC are shown. In contrast to plasmids or synthetic reference templates, cIT-QC cell lines are particularly well suited to be used as control because they are sources of large quantities of genomic DNA and are commercially available. cIT-QC rearrangements represent ⅔ of the amplifiable rearrangement types over all eight primer sets, and thus offer an opportunity to highlight a number of issues, most obviously over-/under-amplification, but also bioinformatic misidentification. Additionally, cIT-QC rearrangements can replace buffy coat DNA for PCR stability without influencing the patient immune repertoire (since cIT-QC rearrangements are bioinformatically identified and by default excluded from the results).

The cIT-QC enables the conversion from reads to cells, which is of utmost importance for clinical use. Diagnostic material being analysed for MRD marker identification can show abundances of particular clonotypes that do not reflect the clonal composition of the sample. For example, if the diagnostic sample is highly infiltrated by a lymphoid malignancy that does not harbour a targetable rearrangement, the (few) residual lymphoid cells would generate the whole spectrum of detectable rearrangements; in such situations minor accompanying physiological B or T cell clones could be misassigned as clones with leukemic markers.

In addition to its use in marker identification, and as exemplarily shown for B and T cell depletion in aplastic follow-up samples, the cIT-QC is of utmost relevance for MRD quantification in samples on or after treatment, in particular if B or T cell directed therapy, which minimises the background of polyclonal gene rearrangements, was applied. If the relative tumor burden is calculated by the ratio of leukemia-specific reads to all annotated reads without any normalisation, the quotient reflects the marker frequency only among cells carrying a particular type of rearrangement (e.g. IG rearrangements in the total pool of B cells present) and might thus heavily overestimate the actual tumor load⁴⁴.

Still further, the QC protocols can be readily embedded in ARResT/Interrogate, which informs users with reports and messages and allows them e.g. to include the QC-failed samples back into the analysis. The logic behind this is that the flag “fail” is an alarm that pre-defined QC criteria were not met, but it does not necessarily indicate that the data are fully corrupt. However, flagged data should always be used with caution, and dependent on the application or question.

The invention therefore also relates to the use of a composition according to the invention or a kit as described herein above in an assay for detecting IG/TR gene rearrangements. A person skilled in the art will recognize and appreciate the diverse range of applications. Only by way of example, the assay is a clinical diagnostic assay, preferably an assay for detecting clonality, identifying MRD markers and/or MRD monitoring and/or analyzing the (clonal) immune repertoire in a lymphoid malignancy.

A further embodiment relates to an in vitro method for detecting IG/TR gene rearrangements in at least one biological sample using NGS, comprising the conventional steps of sample preparation, PCR and/or library construction, sequencing and bioinformatics analysis, but characterized in that at least one of the QC compositions is used. For example, at least one biological sample is spiked with a cIT-QC composition, e.g. in an amount to provide the DNA of at least 40 cell copies of each cell line. Such use as in-tube qualitative and quantitative control enables the conversion from reads to cell correlates, which is of utmost importance for clinical use.

Alternatively or additionally, a cPT-QC composition is run as a separate sample in parallel to the at least one biological “test” sample(s), therewith serving as external control to check the primer and assay performance after sequencing.

Typically, the at least one biological sample is a clinically relevant sample. In one aspect, it is a sample for detection of clonality to support or exclude the diagnosis of malignant lymphoproliferation. In another aspect, it is a sample taken for MRD marker identification or for MRD monitoring analysis or for (clonal) immune repertoire analysis.

A method provided herein can be performed using standard means and protocols known in the art. In one embodiment, at least part of the method is performed using microfluidics technology. For example, the steps of sample preparation, PCR, library construction and/or sequencing is performed in a microfluidics device comprising one or more prestored reagents. Particularly preferred for use in a method of the invention is a centrifugal-microfluidic disk system (also known in the art as “centrifugal microfluidic biochip” or “centrifugal micro-fluidic biodisk”) which is a type of lab-on-a-chip technology that can be used to integrate processes such as separating, mixing, reaction and detecting molecules of nano-size in a single piece of platform, including a compact disk or DVD. There are various typical units in a centrifugal microfluidic structure, including valves, volume metering, mixing and flow switching. These types of units can make up structures that can be used in a variety of ways. Before the molecules react with the reagents, they should be prepared for the reactions. The most typical is separation by centrifugal force. In the case of blood, for example, the sedimentation of blood cells from plasma can be achieved by rotating the biodisk for some time. After separation, all molecular diagnostic assays require a step of cell/viral lysis in order to release genomic and proteomic material for downstream processing. Typical lysis methods include chemical and physical method. The chemical lysis method, which is the simplest way, uses chemical detergents or enzymes to break down membranes. The physical lysis can be achieved by using bead beating system on a disk. Lysis occurs due to collisions and shearing between the beads and the cells and through friction shearing along the lysis chamber walls.

In one aspect, the disk comprises pre-stored reagents for automated and integrated DNA extraction, PCR and/or library generation. See for example the review by Tang et al⁴⁵.

Exemplary disks for use in a method of the invention include those having one or more of the specific features as disclosed in patent application in the name of Hahn Schickard, such as WO2013/124258, WO2014/198703, WO2015/189280, WO2015/051950 and WO2017/191032.

In a method of the invention, the step of bioinformatic analysis advantageously comprises the use of a web-based, interactive application. For example, bioinformatic analysis comprises the use of a purpose-built bioinformatic application (such as ARResT/Interrogate, or equivalent) for the pre-processing of raw data, primer sequence analysis, immunogenetic annotation, post-processing of results, analysis and use of the cIT-QC (including for marker quantification), analysis and use of the cPT-QC (including for comparison to pre-analyzed stored reference datasets), reporting of/access to/visualization of results.

Herewith, the invention demonstrates the applicability of two reference/QC standards, which allow standardised analysis of IG/TR NGS data (e.g. using the NGS primer sets herein disclosed) with high reproducibility, accuracy and precision in marker identification. With ARResT/Interrogate, a complete in silico solution accompanying the in vitro assays is provided, which enables an analysis of IG/TR sequences including all quality criteria and quantification concepts necessary for valid marker identification in lymphoid malignancies.

### LEGEND TO THE FIGURES

FIG. 1. Study design: workflows of development and application for cIT-QC and cPT-QC, and schematic overview of test dataset based on a 96-well plate.

FIG. 2. Schematic overview of the SOP for quality control and quantification in marker identification: library preparation, PCR & NGS, bioinformatics with ARResT/Interrogate.

FIG. 3. Plots of relationships between cIT-QC and markers in the test dataset. A. Relationship between % abundances of reads for cIT-QC and markers (at the x-axis). For cIT-QC, the % denominator is reads with junction; for markers, the % denominator is what we term “usable” reads with junction, which excludes cIT-QC reads; this leads to sums of >100%. B. Abundance of markers before and after normalisation to percentage of cells. *normalisation may lead to values >100%.

FIG. 4: Schematic overview of the workflow for multicentre validation of IG/TR NGS assays for MRD marker identification in ALL. The IG and TR gene rearrangements are amplified in a two-step approach using multiplex PCR assays. Each of the participating laboratories performed NGS-based IG/TR MRD marker identification in 10 patients with ALL. The central polytarget control (cPT-QC) composition of the invention was used to monitor primer performance, and central in-tube quality/quantification controls (cIT-QC) of the invention were spiked to each sample as library-specific quality control and calibrator. Pipetting was performed in a 96-well format. The data analysis was performed employing ARResT/Interrogate.

FIG. 5: Schematic diagrams of rearrangements and primer sets, and histograms showing junctions nucleotide lengths for each investigated locus.

5A-1) Schematic diagrams of IGH-VJ and IGH-DJ rearrangements. The relative position of the VH family primers, DH family primers and consensus JH primers is given according to their most 5′nucleotide upstream (−) or downstream (+) of the involved RSS.
5A-2) Histograms showing junction nucleotide lengths of complete IGH rearrangements (IGH-VJ tube) in a BCP-ALL patient, cPT-QC, BC, thymus, and tonsil. Bars are coloured according to the V-J genes combination.
5A-3) Histograms showing junction nucleotide lengths of incomplete IGH rearrangements (IGH-DJ tube) in a BCP-ALL patient, cPT-QC, BC, thymus, and tonsil. Bars are coloured according to the D-J genes combination.
5B-1) Schematic diagrams of IGK-VJ rearrangement and the two types of Kde rearrangements (V-Kde and intronRSS-Kde). The relative position of the VK, JK, Kde, and intronRSS (INTR) primers is given according to their most 5′nucleotide upstream (−) or downstream (+) of the involved RSS.
5B-2) Histograms showing junction nucleotide lengths of IGK-VJ and IGK-V-Kde rearrangements (IGK-VJ-Kde tube) in a B-ALL patient, cPT-QC, BC, thymus, and tonsil. Bars are coloured according to the V-J-Kde genes combination.
5B-3) Histograms showing junction nucleotide lengths of intron-Kde rearrangements (intron-Kde tube) in a BCP-ALL patient, cPT-QC, BC, thymus, and tonsil.
5C-1) Schematic diagrams of TRB-VJ rearrangement and DJ rearrangement. The relative position of the TRB V family primers, TRB D primers and the TRB J primers is given according to their most 5′nucleotide upstream (−) or downstream (+) of the involved RSS.
5C-2) Histograms showing junction nucleotide lengths of complete TRB rearrangements (TRB-VJ tube) in a T-ALL patient, cPT-QC, BC, thymus, and tonsil. Bars are coloured according to the V-J genes combination.
5C-3) Histograms showing junction nucleotide lengths of incomplete TRB rearrangements (TRB-DJ tube) in a T-ALL patient, cPT-QC, BC, thymus, and tonsil. Bars are coloured according to the D-J genes combination.
5D-1) Schematic diagrams of TRG V-J rearrangement and the relative position of the TRG V and TRG J primers. The relative position of the TRG V primers and the TRG J primers is given according to their most 5′nucleotide upstream (−) or downstream (+) of the involved RSS.
5D-2) Histograms showing junction nucleotide lengths of TRG rearrangements (TRG tube) in a T-ALL patient, cPT-QC, BC, thymus, and tonsil. Bars are coloured according to the V-J genes combination.
5E-1) Schematic diagram of VD-JD, DD-JD, DD-DD, and VD-DD, VD-JA29 rearrangements, showing the positioning of VD, JD, DD, and JA29 primers, all combined in a single tube. The relative position of the Vd, Dd, and Jd primers is indicated according to their most 50 nucleotide upstream (−) or downstream (+) of the involved RSS.
5E-2) Histograms showing junction nucleotide lengths of TRD rearrangements (TRD tube) in a T-ALL patient, cPT-QC, BC, thymus, and tonsil. Bars are coloured according to the V-D-J genes combination.

FIG. 6: Results of multicentre validation of assays for MRD marker identification in ALL. Left hand columns: Index sequences identified by Sanger sequencing. Right hand columns: Index sequences identified by NGS. Darkest colored sections of the columns reflect clonal sequences identified by both methods, lightest colored sections are sequences identified only by the respective method. Median colored sections are clonal sequences identified by both methods, but by NGS with an abundance of <5% after normalization.

### EXPERIMENTAL SECTION

### Example 1: Design and Production of the Central In-Tube Quality/Quantification Control (cIT-QC)

### Sources and Methods

In total, 59 human B (n=30) and T (n=29) lymphoid cell lines were obtained from the American Type Culture Collection (ATCC; www.lgcpromochem-atcc.com, Manassas, VA, USA) and the German Collection of Microorganisms and Cell Cultures GmbH (DSMZ; www.dsmz.de, Braunschweig, Germany), or were derived from internal cell line banks. DNA from cultured cell lines was isolated using a phenol-chloroform extraction protocol, followed by ethanol precipitation and elution in Tris ethylenediaminetetra-acetic acid (TE) buffer. Alternatively, DNA was isolated with the GenElute Mammalian Genomic DNA Miniprep Kit (Sigma-Aldrich, St. Louis, MO, USA) according to manufacturer's protocol.

### Identification of Cell Line-Specific Clonal IG/TR Gene Rearrangements

Each of the 59 cell lines was screened for clonal IG/TR gene rearrangements using the aforementioned EuroClonality-NGS assay with 100 ng of DNA (quantified with Qubit 3.0, Thermo Fisher Scientific) from each cell line, without addition of buffy coat (BC). Paired-end sequencing (2×250 bp) was performed on an Illumina MiSeq (Illumina, San Diego, CA, USA) with a final concentration of 7 pM per library aiming for at least 2000 reads per sample. To avoid low-complexity library issues 10% PhiX control was added to each sequencing run.

### Verification of Cell Line-Specific Clonal IG/TR Gene Rearrangements

Additional methods were used to verify the NGS-amplicon-identified cell line rearrangements:
1. A capture-based protocol, established within EuroClonality-NGS Working Group and covering the coding V, D and J genes of IG/TR loci³⁷: in short, cell line DNA was fragmented and processed with the KAPA Hyperplus Kit with Library Amplification (Roche Sequencing Solutions, Pleasanton, CA, USA); hybridisation of libraries was performed with customised SeqCap EZ Choice Probes (Roche Sequencing Solutions, Pleasanton, CA, USA), developed based on Wren et al³⁷; 2×150 bp paired-end sequencing was performed on Illumina NextSeq.
2. Multiplex amplification and Sanger sequencing according to the BIOMED-2 protocol: PCR products were checked for fragment sizes and clonality in the QIAXCEL Advanced System^{11,46}. Clonal PCR products were subjected to heteroduplex analysis and sequenced on either an ABI 3130 or ABI 3500 platform (Applied Biosystems, Foster City, CA, USA).

IG/TR rearrangement profiles of all cell lines, as obtained with the different methods, were compared.

Verification of Cell Line-Specific Gene Rearrangements from Human B and T Cell Lines Via ddPCR

For cases with discrepant results between the three methods, IG/TR allele-specific PCR assays were designed for digital droplet PCR (ddPCR) (QX200™ Droplet Digital™ PCR System, Bio-Rad) to verify the respective rearrangement. Absolute quantification of IG/TR gene rearrangements by ddPCR was performed using two different gDNA amounts (50 ng, 100 ng). Each experiment included a polyclonal buffy coat BC control and a no template control.

Allele-specific primers for clonal IG/TR rearrangements and probes for quantification were synthesized by Sigma Aldrich. All primers were cleaned by desalting, while hydrolysis probes containing a 5′-FAM/3′-TAMRA reporter dye were cleaned by HPLC. All oligonucleotides were resuspended in TE buffer at a total strand concentration Ct=100 ⋅M and stored at −20° C. before use.

ddPCR reactions were prepared in a volume of 20 ⋅L using 10 ⋅L by 2×ddPCR SuperMix (Bio-Rad Laboratories, Hercules, CA), testing two different amounts of cell line gDNA (50 ng/500 ng) quantified before with the Qubit dsDNA High Sensitivity Assay Kit (Thermo Fisher Scientific, Waltham, MA), forward primer (FP) and reverse primer (RP), each at a final concentration 300 nmol/L, and FAM-labelled probes (100 nmol/L). Droplets were generated by the QX200 droplet generator (Bio-Rad) using 20 ⋅L of the reaction mixture and 70 ⋅L of the droplet generation oil for probes (Bio-Rad), located onto suitable holes in a DG8 cartridge (Bio-Rad). About 45 ⋅L of the drop-oil mixture (12,000-20,000 drops) were transferred to a 96-well plate (Bio-Rad) and loaded on a DNA Engine Dyad Peltier Thermal Cycler with the following amplification protocol: 95° C. for 10 min, followed by 40 cycles: denaturation at 94° C. for 30 s; annealing at 60° C. for 1 min; extension at 60° C. for 1 min. PCR products were loaded into the QX200 droplet reader and analysed by QuantaSoft Version 1.2 (Bio-Rad Laboratories).

### Cell Line Mixture Preparation

Initially, quantification of DNA of selected B- and T-cell lines was done by Qubit dsDNA High Sensitivity Assay Kit (Thermo Fisher Scientific, Waltham, MA). Quantitative values were checked again by ddPCR-based quantification of the albumin housekeeping gene using 50-200 ng DNA/cell line in order to precisely determine the number of cells per μl of DNA. Primers and probe for albumin quantitation were synthesized by Sigma Aldrich. ddPCR was carried out according to the protocol described above, in duplicates for each cell line. After completion of the PCR, samples were analyzed in the Droplet Reader in terms of number of copies of cell lines per 20 μl reaction volume. Based on the values from the ddPCR, the cell line DNA was diluted in TE buffer down to 400 copies/μl. Thereafter, another ddPCR quantification was performed to check the dilution of each cell line DNA again. Two different volumes of the diluted cell line solution (0.5 μl DNA [200 copies] and 2 μl DNA [800 copies]) were used as input amount. With suitable quantitative values, cell line DNAs were further diluted and mixed with each other leading to 40 copies of each cell line being present in 2 μl of the DNA mixture. This mixture was added to each sample as cIT-QC and subjected to simultaneous library preparation prior to sequencing.

Implementation of the cIT-QC

Bioinformatically, cIT-QC reads are identified using an immunogenetic annotation-based approach that is extremely fast while allowing for variations in sequence, avoiding compute-intensive and potentially inaccurate alignment-based approaches. In ARResT/Interrogate, the term ‘spike-ins’ is also used to refer to the cIT-QC.

Regarding QC, identification of at least one read per cIT-QC rearrangement and of at least as many total cIT-QC reads as total cells used is required, otherwise the sample is tagged as “QC-failed” (see below for how this is used in ARResT/Interrogate). The quantification factor—calculated by dividing total cIT-QC cells by total reads—is stored and applied in any case, thus still allowing the user to analyse the sample.

Quantification is based on applying the quantification factor to convert the read counts of a clonotype to cell counts, and then calculate the relative abundance against the total input cells.

### Example 2: Design and Production of the Central Polytarget Quality Control (cPT-QC)

### Sources and Methods

A cPT-QC composition was prepared that consists of genomic DNA isolated from healthy human thymus, healthy human tonsil and healthy human peripheral blood mononuclear cells (DNA amounts mixed in a ratio 1:1:1). To that end, a (semi-)automated genomic DNA extraction was performed on cell suspensions obtained after dissecting and mincing tissues or Ficoll density blood separation.

The cPT-QC composition is suitable used to undergo NGS library preparation alongside the investigated samples. For the EuroClonality-NGS assay, this involves one cPT-QC sample per run, amplified in eight tubes.

Implementation of the cPT-QC

Primers are bioinformatically identified in the reads of each of the eight cPT-QC tubes of the run and their abundances compared to stored cPT-QC reference results using the test of proportions.

Stored reference results are the output of ARResT/Interrogate from the analysis of a cPT-QC sample. These results should be confirmed through replicate runs over time in each lab to accommodate for technical variability. The results and not the raw data are stored to ensure that the bioinformatic analysis is not compromised inadvertently by the user; this means that the results are updated with every major release of ARResT/Interrogate to ensure compatibility with new runs.

Issues with abundances of particular primers or a specific primer set are used to tag the corresponding cPT-QC samples plus all user samples of the same primer set as “QC-failed”.

### Replicate Runs

As reproducibility is important for a QC of this type, replicate runs of the cPT-QC were performed. Relative abundances of 5′ primers were compared employing the test of proportions.

### Primer Perturbation Runs

To assess the usability of the cPT-QC to detect problems with primer performance, artificial perturbations of primer concentrations were created to simulate missing pipetting a primer or pipetting the wrong primer concentration.

First the 5′ primer usage was analysed in a cPT-QC sample and two primers of differing abundances were selected from each primer set, thereby skipping the intron-Kde primer set, which only has two primers; IGH-VJ-FR1-M-1, IGH-V-FR1-O-1; IGH-D-B-1, IGH-D-E-1; IGK-V-G-1, IGK-V-I-1; TRB-V-AD-1, TRB-V-G-1; TRB-D-A-1, TRB-D-B-1; TRG-V-F-1, TRG-V-E-1; TRD-D-A-1, TRD-V-B-1. Those primers were perturbed by fully excluding them from the primer pool and changing their concentration by reduction to 10% and increase to 200%. Relative abundances of 5′ primers were compared between these perturbed sets and cPT-QC employing the test of proportions.

### Creation of a Test Dataset

A dataset was created to evaluate and showcase the aforementioned concepts and functionalities, which consists of the following samples:
1. Four diagnostic bone marrow B-/T-ALL samples with high leukemic cell content (leukemic infiltration assessed by routine cytomorphology to be 60-80%).
2. Four samples of patients with B/T cell aplasia after B/T cell targeted treatment. The two samples with B cell aplasia were CLL samples after Rituximab (anti-CD20) treatment and the two samples with T cell aplasia were T-PLL (prolymphocytic leukemia) samples after Alemtuzumab (anti-CD52) treatment. In all these samples lineage-specific aplasia was confirmed by flow cytometry.
3. cPT-QC for all IG/TR primer sets, but with the TRB-VJ primer set results swapped with perturbed results from validation experiments as outlined above. To showcase the generic QC functionalities, <1000 random reads from one of the diagnostic samples were artificially chosen.

### Methodology

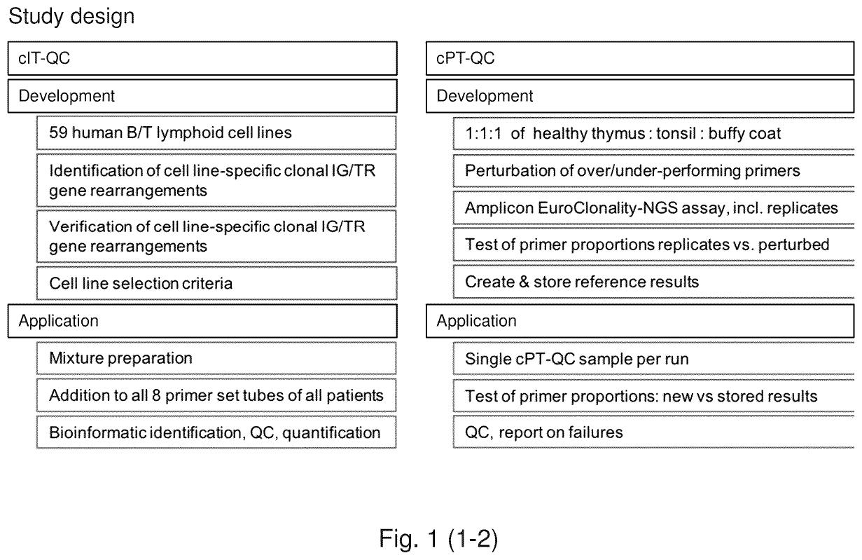

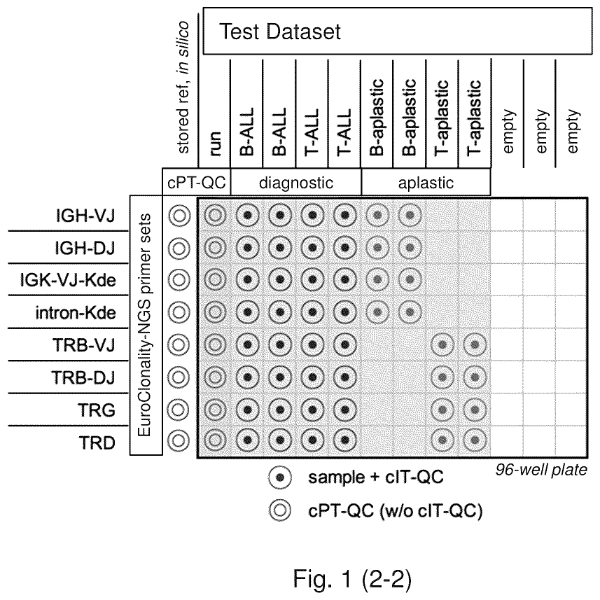

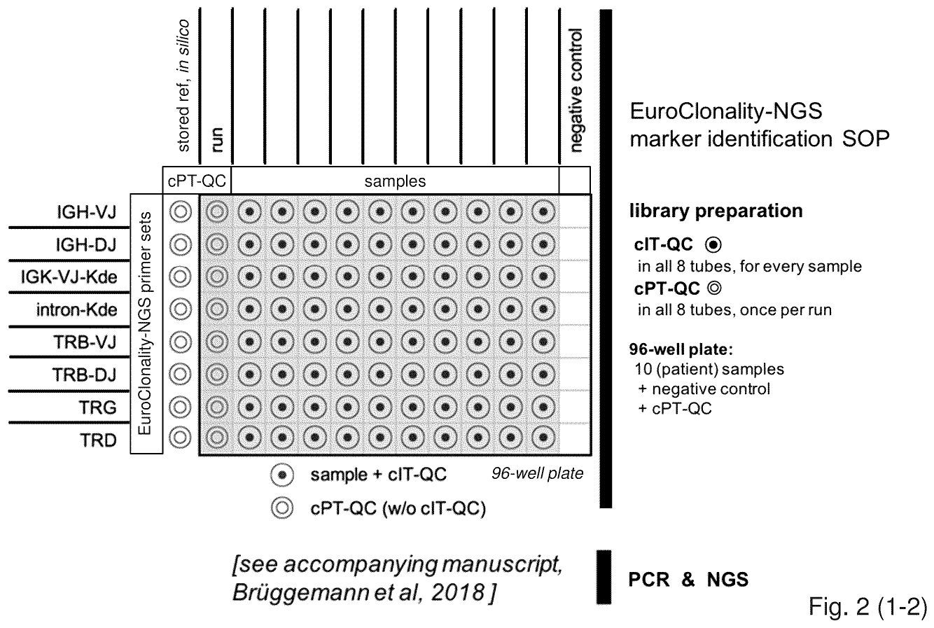

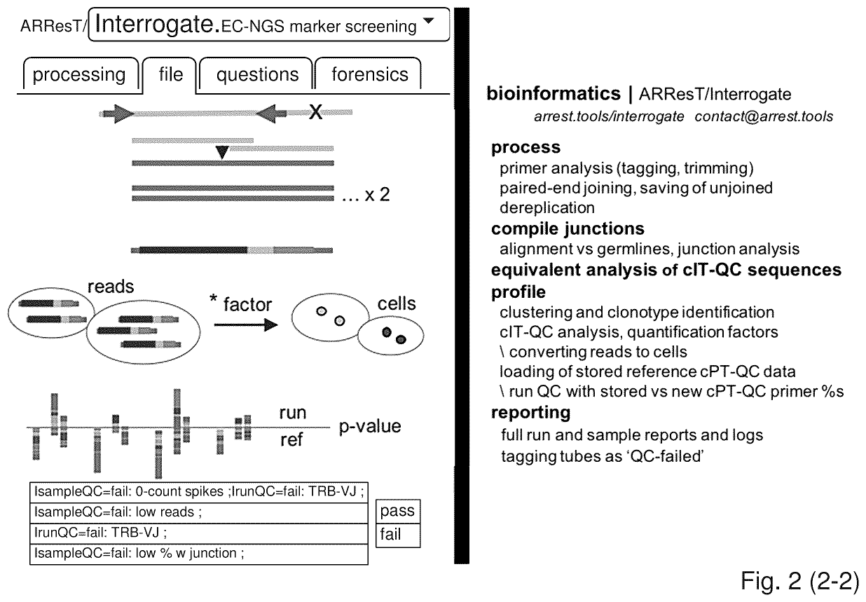

The diagnostic samples and the cPT-QC were run with all primer sets, while the aplastic follow-up samples were only run with the corresponding primer sets, i.e. the IG sets for samples with B cell aplasia, and the TR sets for samples with T cell aplasia (as depicted in FIG. 1; test dataset). Additionally, the follow-up samples were run without addition of buffy coat (BC) to test if the addition of the cIT-QC composition is sufficient to stabilise the samples for sequencing, without compromising their immunogenetic profile. To this end, the protocol visualised in FIG. 2 was followed.

Primer Performance Assessment Using the cPT-QC

The tests of proportions of 5′ primer relative abundances, applied to the cPT-QC, BC, their replicates, and to the libraries with primer perturbations, showed that there is a clear difference in p-values between sets of un-perturbed and perturbed primers. In other words, the p-values of the differences in abundance of the perturbed primers are noticeably lower. Table 2 presents a simplified view of the results, focusing on the abundances of perturbed primers plus at least one other un-perturbed primer per primer set either to show their normal behavior or discuss their abnormal behavior. Percentage abundances of 5′ primers across all primer sets. Top group of primers were perturbed; bottom group is a selection of primers that were left un-perturbed: one per primer set selected alphabetically, plus two examples where the primer behavior is of interest to the discussion (see text). Results are shown: in cPT-QC replicates (third column); against samples where primers were excluded (“0%”, fourth column), reduced to 10% (fifth column), increased to 200% (sixth column). Changes in percentages (indicated separately and as +/−) that led to the test of proportions.

At a p-value threshold of 1e-200, none of the primers are flagged in the cPT-QC, which highlights the reproducibility of the assay, while all the perturbed primers are flagged in the perturbed scenarios. In fact, the lowest p-value in the normal samples is 7.86e-142 for primer TRD-V-A-1 (Table 2), compared to multiple zero values in the perturbed comparisons (with a few exceptions, mainly for the 200% perturbation). Significant changes in abundance were also visible in other cells, with the most likely explanation that those primers were indirectly affected by perturbations of other primers. That is, a primer “taking over” when an initially abundant primer was excluded, such as IGH-V-FR1-D-1 when IGH-VJ-FR1-M-1 is perturbed either way especially since these primers amplify partially overlapping lists of genes.

### Evaluation of QC Aspects in ARResT/Interrogate

Information on the in silico quality control based on both the cPT-QC and cIT-QC is available in ARResT/Interrogate, with “QC-failed” samples excluded by default to warn and prevent the user from their unintended use. However, the user is notified and has the option to include them back in the analysis.

Generic quality control is also performed on samples, specifically to check for low number of raw reads and low percentage of reads with an identified junction. Such samples are also tagged as “QC-failed”.

TABLE 2

| primers | | | cPT-QC | | | cPT-QC vs 0% | | cPT-QC vs 0% | | cPT-QC vs 200% | |
|---|---|---|---|---|---|---|---|---|---|---|---|
| primer set | | primer name | rep1 | <diff> | rep2 | <diff | rep1 | <diff | rep1 | <diff | rep1 |
| IGH-VJ-FR1 | perturbed | IGH-V-FR1-M-1 | 27.44 | −5.2 | 22.24 | −26.71 | 0.7297 | −25.42 | 2.017 | +7.72 | 35.16 |
| IGH-VJ-FR1 | | IGH-V-FR1-0-1 | 1.184 | −0.089 | 1.095 | −1.121 | 0.06314 | −1.116 | 0.06792 | +1.681 | 2.865 |
| IGH-DJ | | IGH-D-B-1:#1:14C | 7.318 | +0.12 | 7.438 | −7.318 | 0 | −7.271 | 0.0474 | +7.152 | 14.47 |
| IGH-DJ | | IGH-D-B-1:#2:14T | 11.74 | +0.48 | 12.22 | −11.74 | 0.0008552 | −11.65 | 0.08643 | +11.48 | 23.22 |
| IGH-DJ | | IGH-D-E-1:#4:14G22G | 1.664 | −0.099 | 1.765 | −1.859 | 0.005364 | −1.853 | 0.01096 | −0.2 | 1.664 |
| IGK-VJ-Kde | | IGK-V-G-1 | 8.08 | +0.169 | 6.249 | −5.954 | 0.1259 | −5.911 | 0.169 | +7.51 | 13.59 |
| IGK-VJ-Kde | | IGK-V-I-1 | 8.648 | +0.057 | 8.905 | −8.789 | 0.0588 | −8.495 | 0.3527 | +11.86 | 20.71 |
| TRB-VJ | | TRB-V-AD-1 | 31.76 | +1.88 | 33.64 | −31.41 | 0.3514 | −28.86 | 4.905 | +3.93 | 35.69 |
| TRB-VJ | | TRB-V-G-1 | 10.09 | −0.515 | 9.575 | −10.06 | 0.02769 | −9.889 | 0.2006 | +1.76 | 11.85 |
| TRB-DJ | | TRB-D-A-1 | 63.2 | +0.95 | 64.15 | −63.19 | 0.01025 | −48.89 | 14.31 | +6.53 | 69.73 |
| TRB-DJ | | TRB-D-B-1 | 36.14 | −1.36 | 34.78 | −36.06 | 0.0784 | −33.22 | 2.919 | +12.71 | 48.85 |
| TRD | | TRD-V-B-1 | 12.55 | +2.33 | 14.88 | −12.49 | 0.06142 | −12.14 | 0.4108 | +30.74 | 43.29 |
| TRD | | TRB-D-A-1 | 84.8 | +8.36 | 70.96 | −64.51 | 0.0914 | −62.44 | 2.182 | −7.41 | 57.19 |
| TRG | | TRG-V-E-1 | 3.515 | −0.113 | 3.402 | −3.512 | 0.003061 | −3.455 | 0.05982 | +5.547 | 9.062 |
| TRG | | TRG-V-F-1 | 14.48 | −0.08 | 14.4 | −14.37 | 0.1087 | −14.45 | 0.02861 | +9.05 | 23.53 |
| IGH-VJ-FR1 | | IGH-V-FR1-A-1 | 15.34 | +1.7 | 17.04 | −0.89 | 14.45 | −3.65 | 11.69 | +7.41 | 22.75 |
| IGH-VJ-FR1 | | IGH-V-FR1-D-1 | 16.41 | −1.62 | 14.79 | +26.18 | 42.59 | +22.64 | 39.05 | −9.999 | 6.411 |
| IGH-DJ | | IGH-D-A-1:#1:6C | 8.291 | +1.512 | 9.803 | +1.779 | 10.07 | +1.258 | 9.549 | −0.508 | 7.783 |
| IGK-VJ-Kde | | IGK-V-A-1 | 9.787 | +0.08 | 9.867 | +3.863 | 13.65 | +3.403 | 13.19 | +0.147 | 9.934 |
| TRB-VJ | | TRB-V-AB-1 | 1.423 | +0.054 | 1.477 | +1.48 | 2.903 | +0.517 | 1.94 | −0.069 | 1.354 |
| TRD | | TRD-V-A-1 | 14.37 | −7.114 | 7.256 | +9.44 | 23.81 | +8.12 | 22.49 | −4.508 | 9.862 |
| TRG | | TRG-V-A-1 | 18.71 | +1.7 | 20.41 | +3.06 | 21.77 | +1.9 | 20.61 | −2.63 | 16.08 |

cPT-QC: replicates and primer perturbations.
all numbers % abundances;
rep: replicate;
test of productions vs cPT-QC rep1,
dark grey: <1e−199, light grey: <1e−99

### Example 3: Marker Identification & Quantification

Abundances of lymphocyte subpopulations are frequently not available for samples of patients with lymphoid malignancies. Furthermore, as IG/TR NGS only reflects relative representation of the rearrangements, it was important to establish a calibrator, which would allow to normalise sequencing reads to input DNA cells. This is particularly important for tubes that exclusively cover rearrangements being present only in a minority of lymphoid cells (especially the TRD and intron-Kde tubes). TRD genes are not rearranged in normal B cells and are deleted in most TR⋅⋅ cells. Therefore, oligoclonal TCR⋅⋅ T-cells might give rise to dominant clonotypes in the TRD NGS assay, in particular as the normal TCR⋅⋅ T cell repertoire is strikingly skewed during childhood. Here the cIT-QC-based abundance correction is of utmost importance to avoid miss-assignment of (minor) clonal TRD rearrangements from minor TCR⋅⋅ cell populations as leukemic rearrangements that would then serve as markers in further MRD analysis.

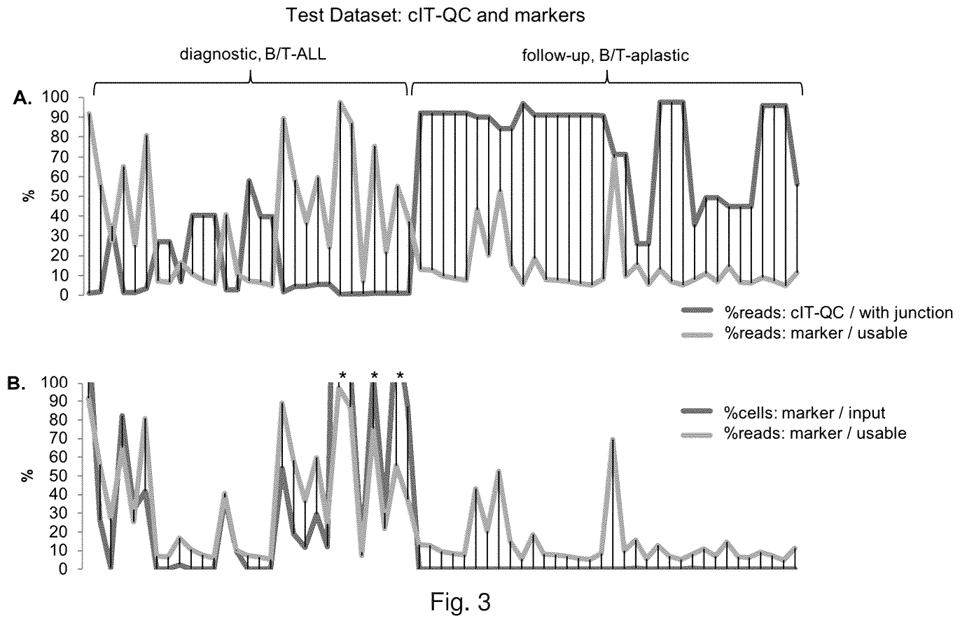

Analysis of the test dataset showed the utility of the cIT-QC in marker identification and quantification. Without the cIT-QC, both diagnostic and aplastic samples seem to be oligoclonal if simply based on the number of reads (FIG. 3). However, the very high number of reads from only a very limited number of cIT-QC cells (120-440, dependent on number of cIT-QC rearrangements per primer set), in all aplastic and a few of the diagnostic samples, are an indirect, yet clear indication of the restricted numbers of patient-related input cells harbouring rearrangements of the particular IG/TR locus in those samples. From another perspective, the total read percentage of cIT-QC is much greater than those of rearrangements in these samples, suggesting that also the number of cIT-QC cells is greater than the number of patient-related input cells. Indeed, after quantification with the cIT-QC, cell abundances fall well below the thresholds implying clonality.

On the other hand, and as expected, in the diagnostic samples cIT-QC sequences constitute a minority. Hence, this implies that with the cIT-QC the abundance of a certain rearrangement can much more accurately be determined and recalculated to cell abundances.

Additionally, five experienced EuroMRD ALL reference laboratories performed IG/TR NGS in 50 diagnostic ALL samples, and compared results with those generated through routine IG/TR Sanger sequencing. A cPT-QC composition was used to monitor primer performance, and a cIT-QC composition was spiked into each sample as a library-specific quality control and calibrator. NGS identified 259 (average 5.2/sample, range 0-14) clonal sequences vs. Sanger-sequencing 248 (average 5.0/sample, range 0-14). The overall concordance between Sanger and NGS, including negative libraries, was 78%.

### Example 4: Development and Multicentre Validation of IG/TR NGS Assays for MRD Marker Identification in ALL

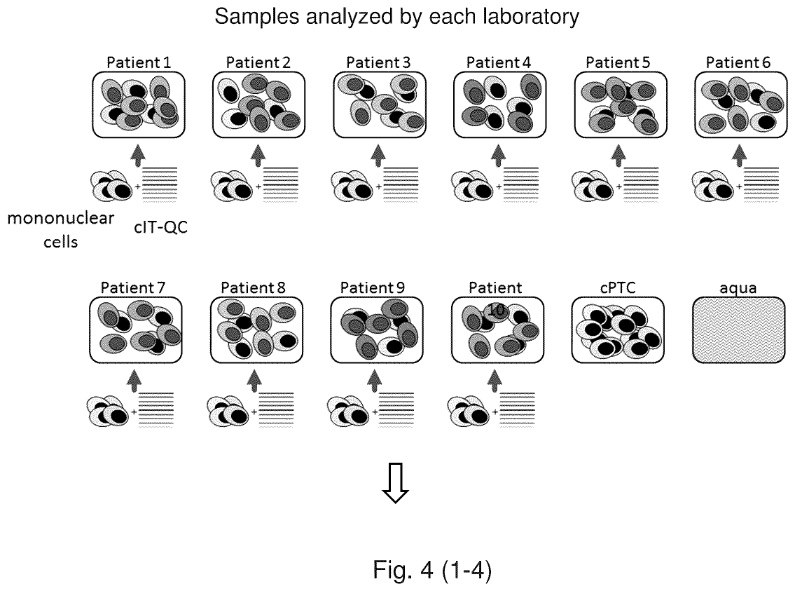

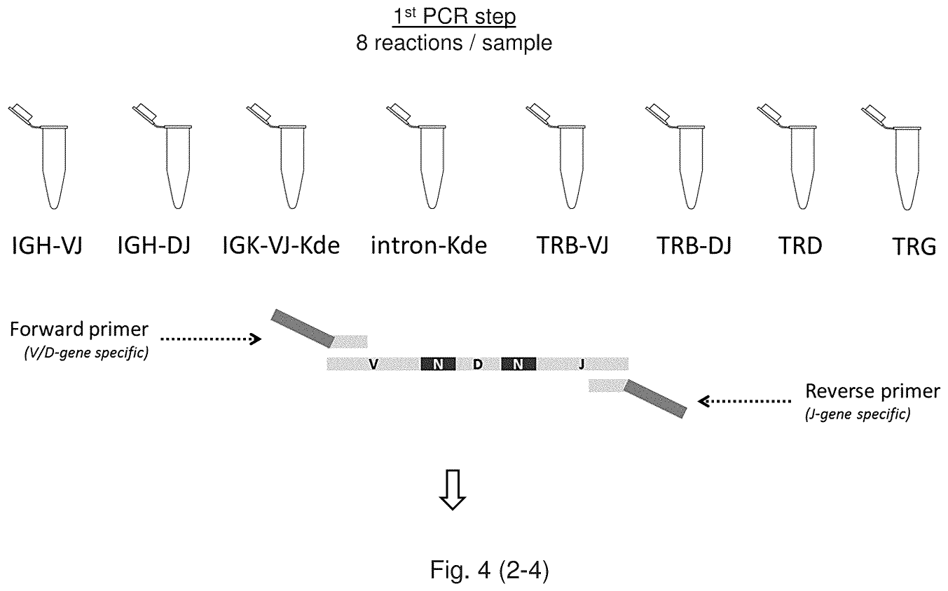

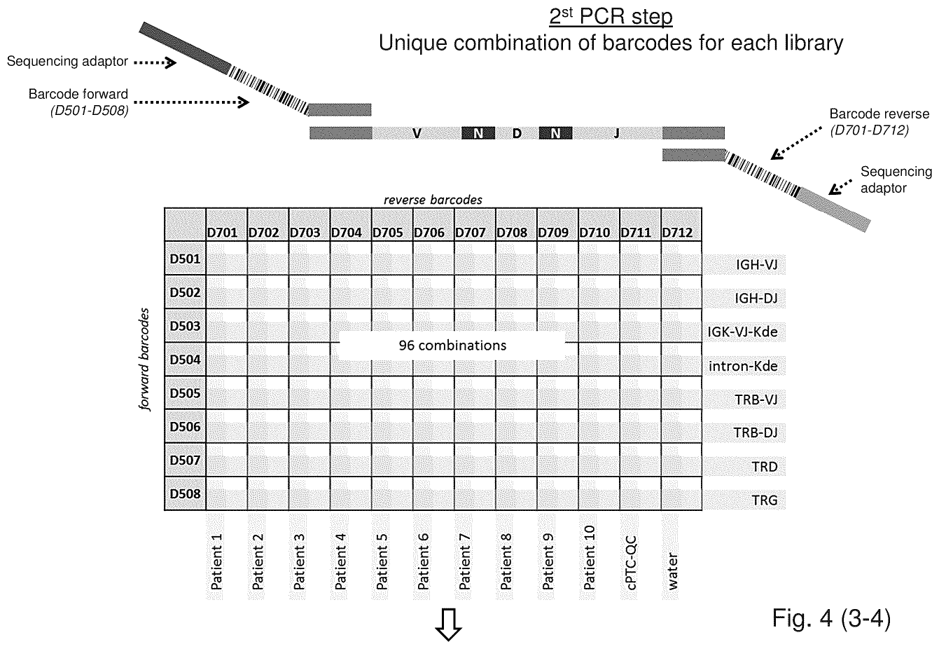

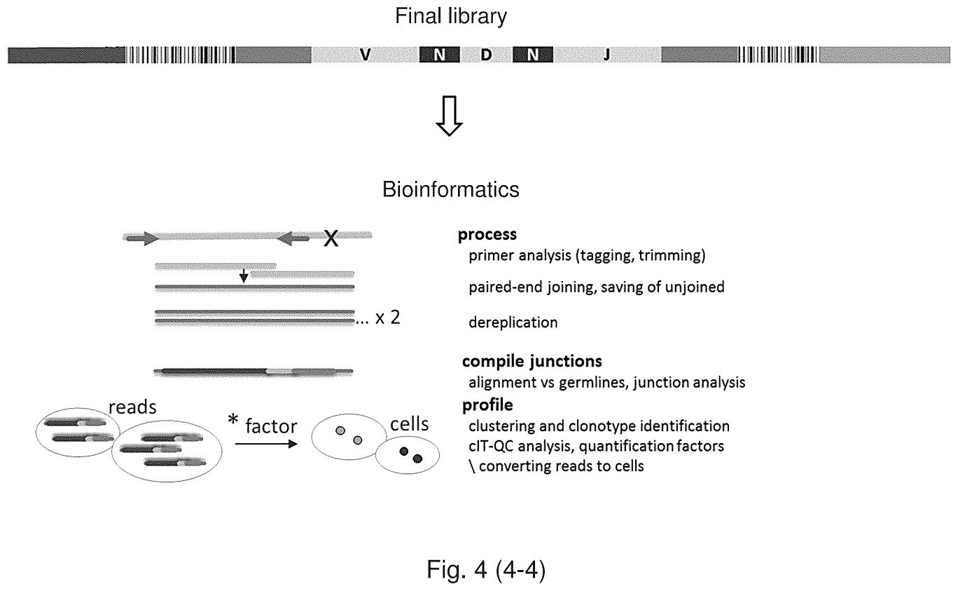

This example describes the development and design of an IG/TR assay, including bioinformatics, and its validation for MRD marker identification in acute lymphoblastic leukemia (ALL). Five EuroMRD ALL MRD reference laboratories performed IG/TR NGS in 50 diagnostic ALL samples, and compared results with those generated through routine IG/TR marker screening and Sanger sequencing. A cPT-QC composition was used to monitor primer performance, and a cIT-QC composition was spiked into each sample as a library-specific quality control and calibrator. The overall workflow of the validation study is shown in FIG. 4.

### Materials and Methods

### General Concept of Assay Design

With the objective of developing a universal amplicon-based NGS approach for IG/TR sequence analysis at the DNA level, applicable in all lymphoid malignancies, assays for multiple IG/TR loci were designed for: IG heavy (IGH), IG kappa (IGK), TR beta (TRB), TR gamma (TRG), and TR delta (TRD), including complete and incomplete rearrangements whenever applicable. IG lambda (IGL) was excluded due to its limited complementarity to other IG loci and its reduced diversity. TR alpha (TRA) was excluded due to its high complexity, hampering a reasonable multiplex PCR approach at the DNA level.

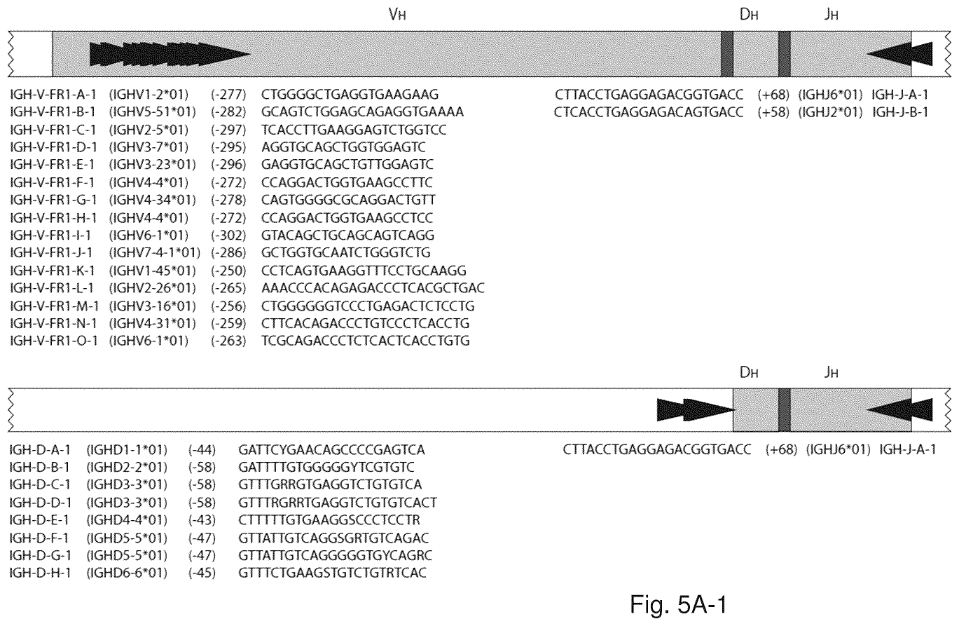

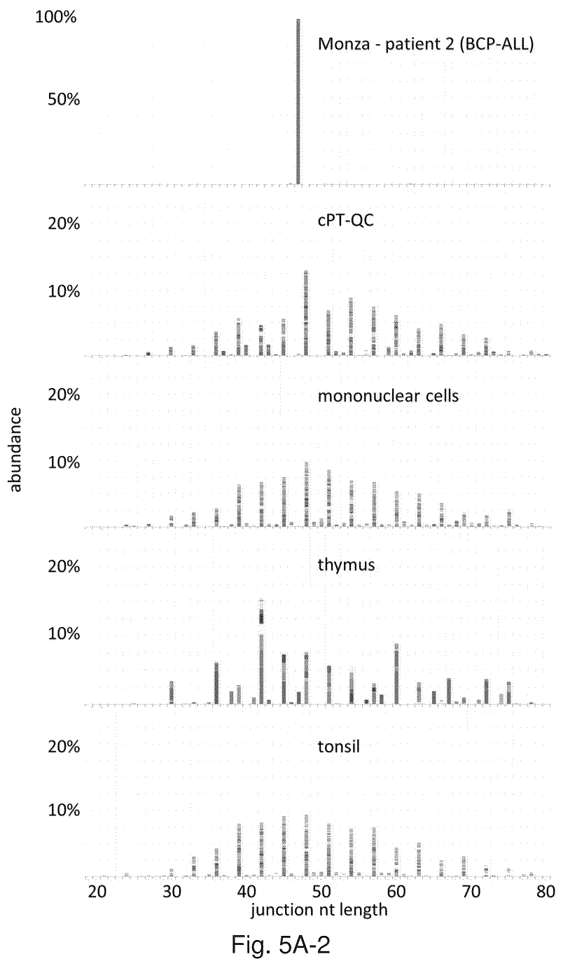

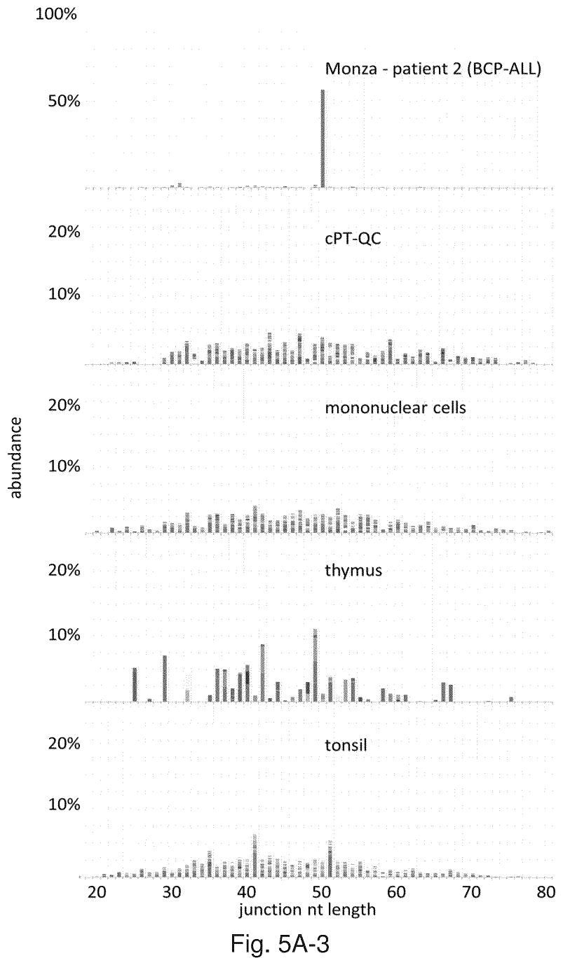

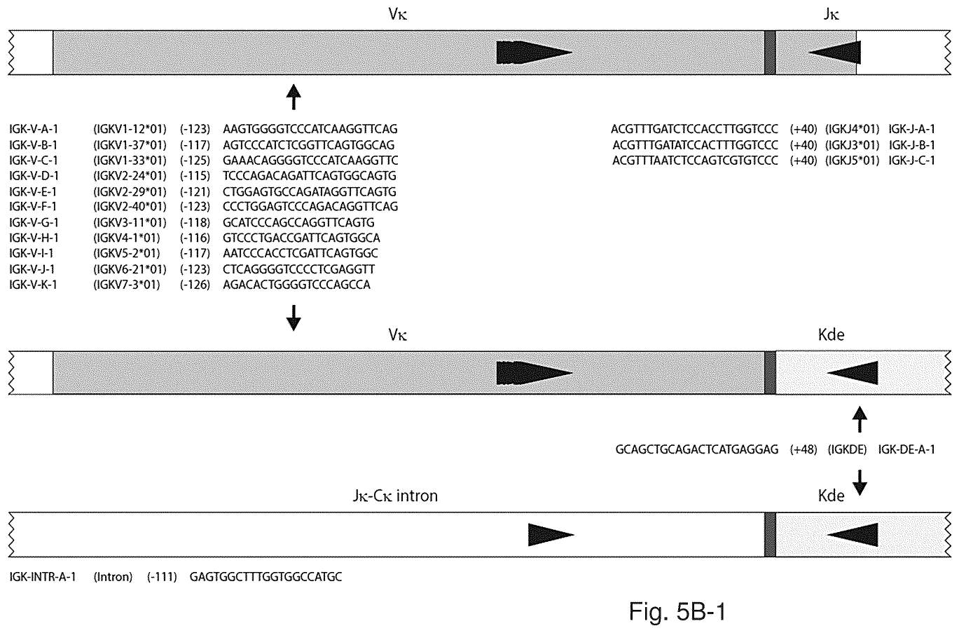

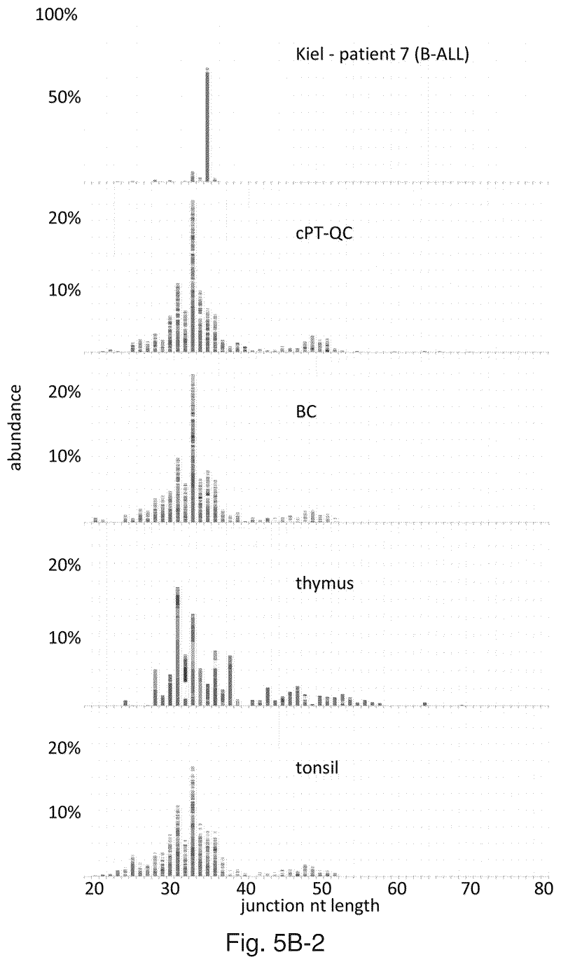

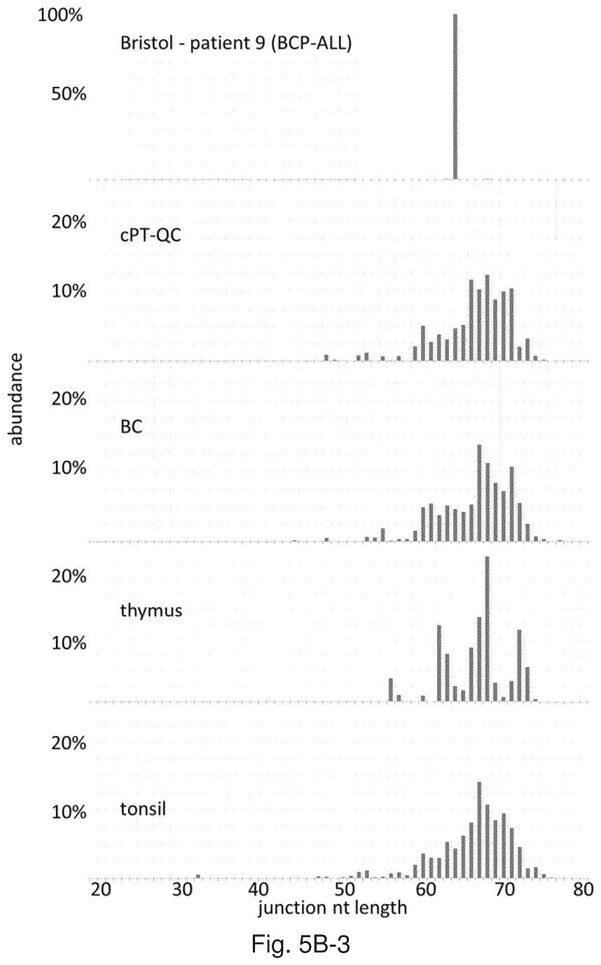

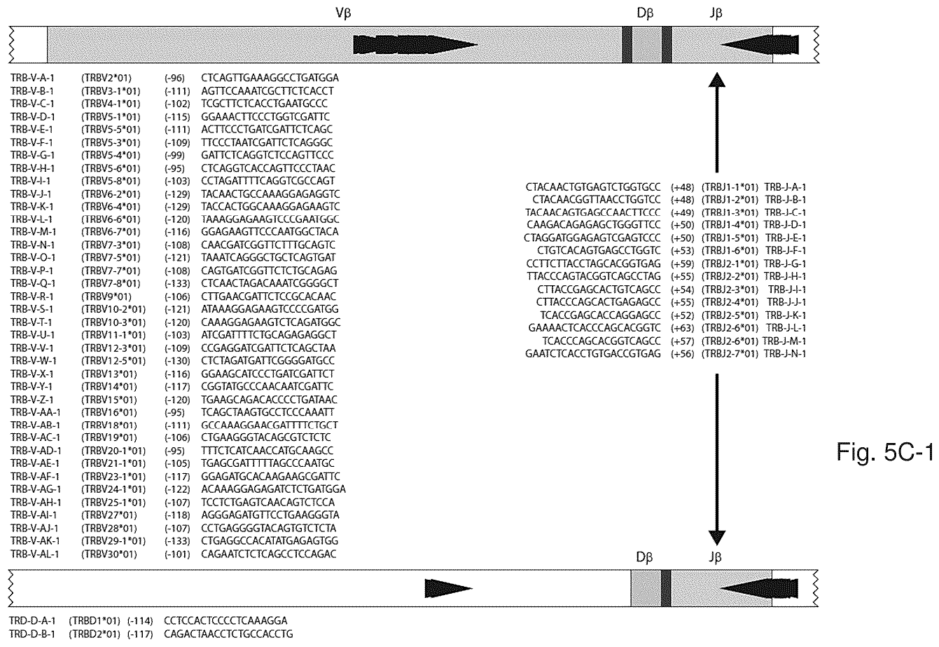

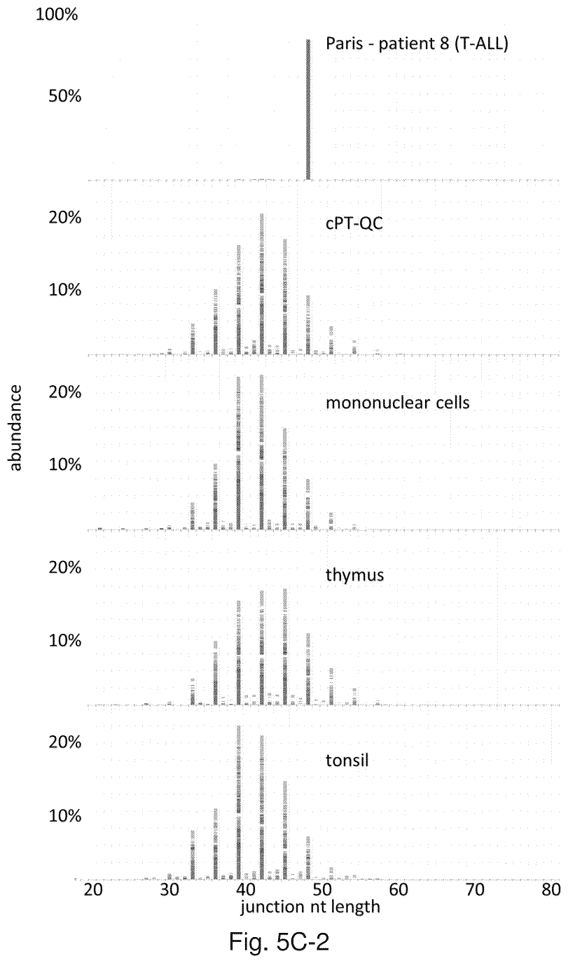

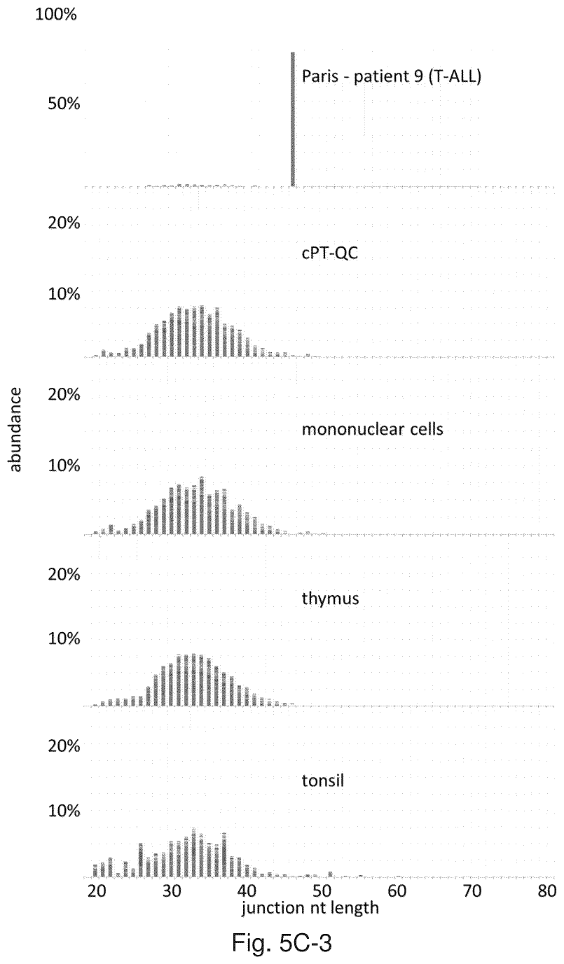

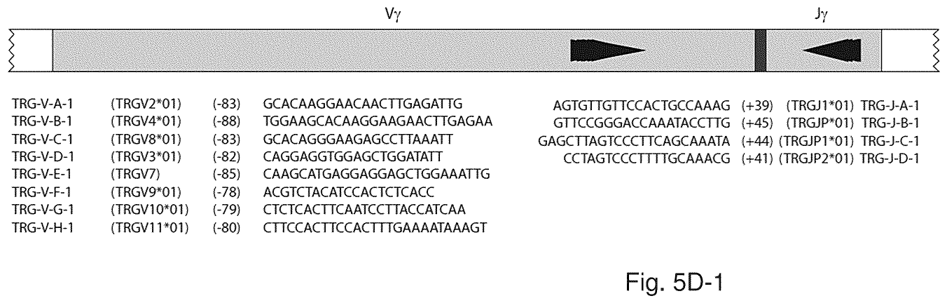

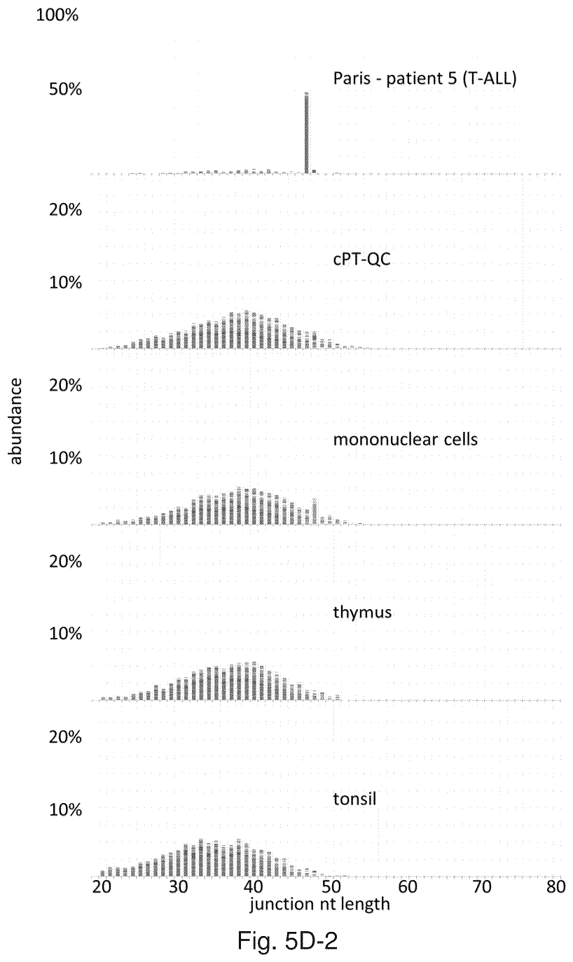

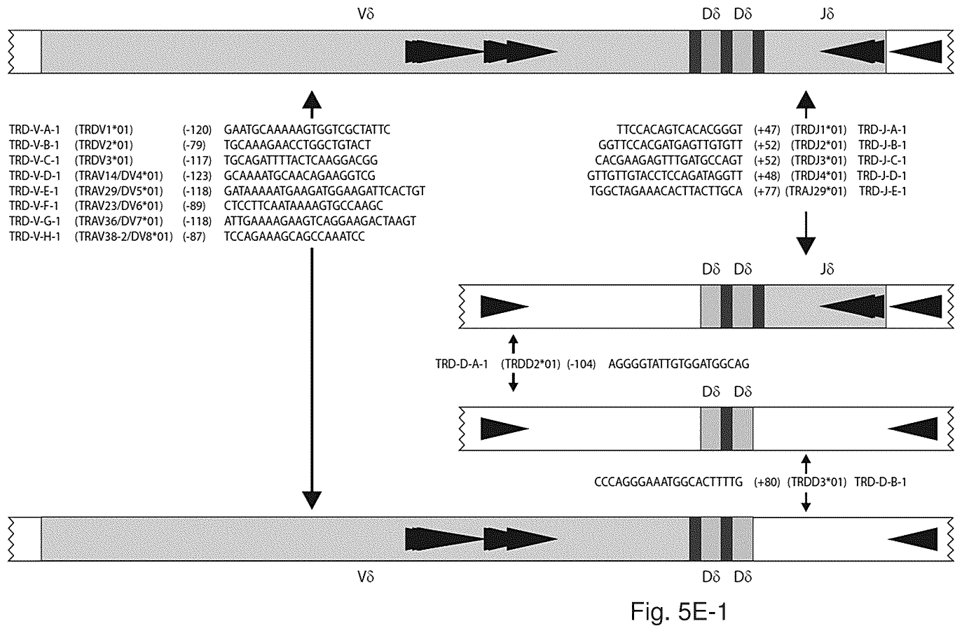

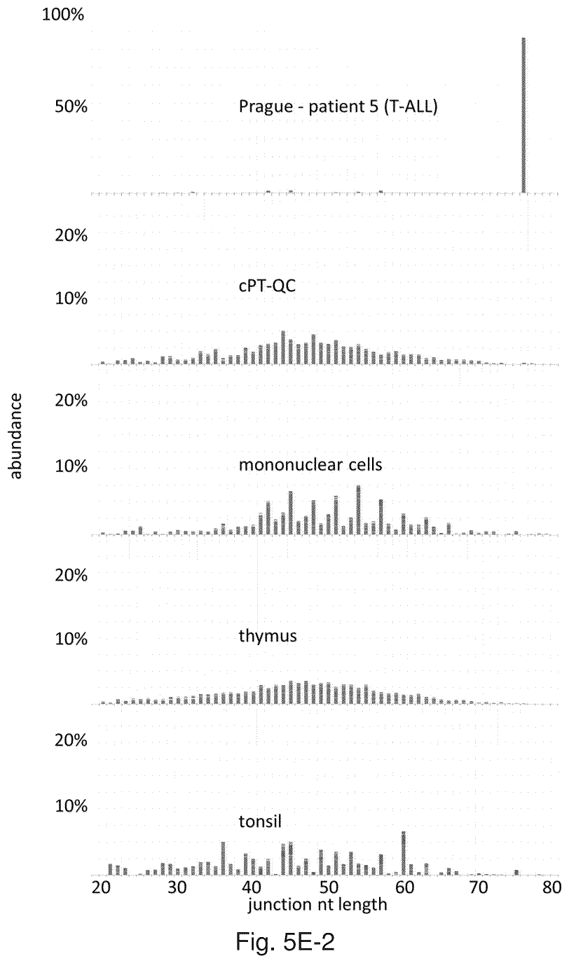

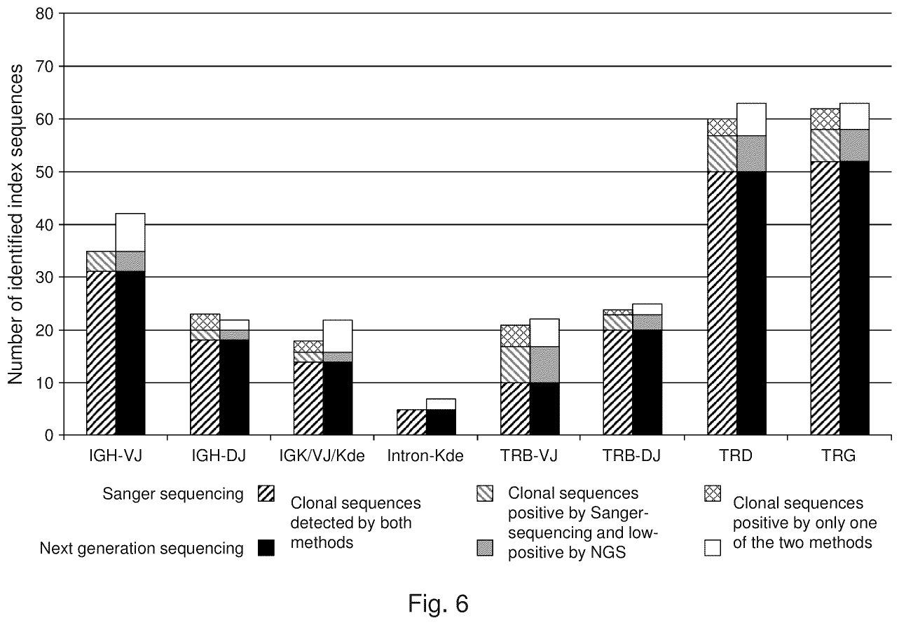

The IGH locus is rearranged in two steps (FIG. 5A). After initial coupling of a single IGH-D gene to an IGH-J gene, an IGH-V gene is joined to the incomplete IGH-DJ rearrangement, resulting in a complete IGH-VJ rearrangement. For amplification of complete IGH rearrangements, primers located in the FR1, FR2 and FR3 regions were designed, but here we only describe the FR1 assay for marker identification in ALL. IGH-DJ rearrangements were amplified in a separate multiplex PCR reaction. The IGK light chain locus is composed of functional IGKV and IGKJ genes, as well as the so-called kappa deleting element (Kde) that can rearrange to IGKV genes, or to a recombination signal sequence (RSS) in the IGKJ-IGKC intron, leading to functional inactivation of the IGK allele (FIG. 5B). The IGKV forward primers were designed to be used in combination with IGKJ and Kde reverse primers in one multiplex reaction, whereas a second PCR was developed for the forward intron RSS and reverse Kde primers.

The TRB locus also features a two-step process with initial formation of incomplete TRB-DJ rearrangements followed by complete TRB-VJ rearrangements. Incomplete and complete TRB rearrangements were designed to be detected in two separate multiplex PCR reactions (FIG. 5C). As TRG locus rearrangements are one-step VJ recombinations involving a limited number of TRGV and TRGJ genes, a single multiplex assay could be developed (FIG. 5D). Finally, in the TRD locus, complete VJ rearrangements are preceded by DD, VD and DJ rearrangements. In addition, certain TRAV genes can rearrange to both TRDJ and TRAJ, whereas TRDV-TRAJ rearrangements, usually involving TRAJ29, can also occur. All of these rearrangements were designed to be amplified in one multiplex PCR assay (FIG. 5E). The bioinformatic platform ARResT/Interrogate⁴³, already developed from the ground-up within the EuroClonality-NGS working group to assist with its multi-faceted activities, was further adapted for this study as described below.

### Primer Design and Technical Validation of Primer Performance

Primers were designed to be gene-specific, but in case of allelic variants, degenerate primers were designed to facilitate multiplexing. For the same reason, single mismatches in the middle or at the 5′-end of the primer were accepted. Table 3 shows the primer sequences comprising nucleotide sequences of FIG. 5 and additional adapter sequences (forward or reverse). Those of skill in the art will realize that the Example is only illustrative and that many variations of the specific methods of the Example are possible. For example, there is no need to use the M13 sequences as part of the primers as used in the Example. This could be replaced by any other known sequence of DNA.

TABLE 3
Primer sequences for the 1^{st} and 2^{nd} step PCR in IG/TR NGS.

| 1^{st} step | | | | |
|---|---|---|---|---|
| PCR | Primer | | Primer | Primer sequence with universal primer sequences |
| tube | nomenclature | μM in PCR | direction | attached (forward/reverse) 5′ to 3′ |
| TRB V-J | TRB-V-C-1 | 0.00625 μM | 5′ | GTAAAACGACGGCCAGTTCGCTTCTCACCTGAATGCCC |
| | TRB-V-A-1 | 0.0125 μM | 5′ | GTAAAACGACGGCCAGTCTCAGTTGAAAGGCCTGATGGA |
| | TRB-V-X-1 | 0.0125 μM | 5′ | GTAAAACGACGGCCAGTGGAAGCATCCCTGATCGATTCT |
| | TRB-V-AA-1 | 0.0125 μM | 5′ | GTAAAACGACGGCCAGTTCAGCTAAGTGCCTCCCAAATT |
| | TRB-V-B-1 | 0.025 μM | 5′ | GTAAAACGACGGCCAGTAGTTCCAAATCGCTTCTCACCT |
| | TRB-V-F-1 | 0.025 μM | 5′ | GTAAAACGACGGCCAGTTTCCCTAATCGATTCTCAGGGC |
| | TRB-V-J-1 | 0.025 μM | 5′ | GTAAAACGACGGCCAGTTACAACTGCCAAAGGAGAGGTC |
| | TRB-V-L-1 | 0.025 μM | 5′ | GTAAAACGACGGCCAGTTAAAGGAGAAGTCCCGAATGGC |
| | TRB-V-M-1 | 0.025 μM | 5′ | GTAAAACGACGGCCAGTGGAGAAGTTCCCAATGGCTACA |
| | TRB-V-S-1 | 0.025 μM | 5′ | GTAAAACGACGGCCAGTATAAAGGAGAAGTCCCCGATGG |
| | TRB-V-W-1 | 0.025 μM | 5′ | GTAAAACGACGGCCAGTCTCTAGATGATTCGGGGATGCC |
| | TRB-V-Z-1 | 0.025 μM | 5′ | GTAAAACGACGGCCAGTTGAAGCAGACACCCCTGATAAC |
| | TRB-V-AE-1 | 0.025 μM | 5′ | GTAAAACGACGGCCAGTTGAGCGATTTTTAGCCCAATGC |
| | TRB-V-AG-1 | 0.025 μM | 5′ | GTAAAACGACGGCCAGTACAAAGGAGAGATCTCTGATGGA |
| | TRB-J-A-1 | 0.025 μM | 3′ | TAATACGACTCACTATAGGGCTACAACTGTGAGTCTGGTGCC |
| | TRB-J-B-1 | 0.025 μM | 3′ | TAATACGACTCACTATAGGGCTACAACGGTTAACCTGGTCC |
| | TRB-J-C-1 | 0.025 μM | 3′ | TAATACGACTCACTATAGGGTACAACAGTGAGCCAACTTCCC |
| | TRB-J-D-1 | 0.025 μM | 3′ | TAATACGACTCACTATAGGGCAAGACAGAGAGCTGGGTTCC |
| | TRB-J-E-1 | 0.025 μM | 3′ | TAATACGACTCACTATAGGGCTAGGATGGAGAGTCGAGTCCC |
| | TRB-J-F-1 | 0.025 μM | 3′ | TAATACGACTCACTATAGGGCTGTCACAGTGAGCCTGGTC |
| | TRB-J-G-1 | 0.025 μM | 3′ | TAATACGACTCACTATAGGGCCTTCTTACCTAGCACGGTGAG |
| | TRB-J-H-1 | 0.025 μM | 3′ | TAATACGACTCACTATAGGGTTACCCAGTACGGTCAGCCTAG |
| | TRB-J-I-1 | 0.025 μM | 3′ | TAATACGACTCACTATAGGGCTTACCGAGCACTGTCAGCC |
| | TRB-J-J-1 | 0.025 μM | 3′ | TAATACGACTCACTATAGGGCTTACCCAGCACTGAGAGCC |
| | TRB-J-K-1 | 0.025 μM | 3′ | TAATACGACTCACTATAGGGTCACCGAGCACCAGGAGCC |
| | TRB-J-N-1 | 0.025 μM | 3′ | TAATACGACTCACTATAGGGGAATCTCACCTGTGACCGTGAG |
| | TRB-V-D-1 | 0.05 μM | 5′ | GTAAAACGACGGCCAGTGGAAACTTCCCTGGTCGATTC |
| | TRB-V-N-1 | 0.05 μM | 5′ | GTAAAACGACGGCCAGTCAACGATCGGTTCTTTGCAGTC |
| | TRB-V-O-1 | 0.05 μM | 5′ | GTAAAACGACGGCCAGTTAAATCAGGGCTGCTCAGTGAT |
| | TRB-V-P-1 | 0.05 μM | 5′ | GTAAAACGACGGCCAGTCAGTGATCGGTTCTCTGCAGAG |
| | TRB-V-R-1 | 0.05 μM | 5′ | GTAAAACGACGGCCAGTCTTGAACGATTCTCCGCACAAC |
| | TRB-V-V-1 | 0.05 μM | 5′ | GTAAAACGACGGCCAGTCCGAGGATCGATTCTCAGCTAA |
| | TRB-V-AB-1 | 0.05 μM | 5′ | GTAAAACGACGGCCAGTGCCAAAGGAACGATTTTCTGCT |
| | TRB-V-AI-1 | 0.05 μM | 5′ | GTAAAACGACGGCCAGTAGGGAGATGTTCCTGAAGGGTA |
| | TRB-V-AJ-1 | 0.05 μM | 5′ | GTAAAACGACGGCCAGTCCTGAGGGGTACAGTGTCTCTA |
| | TRB-V-AL-1 | 0.05 μM | 5′ | GTAAAACGACGGCCAGTCAGAATCTCTCAGCCTCCAGAC |
| | TRB-V-E-1 | 0.1 μM | 5′ | GTAAAACGACGGCCAGTACTTCCCTGATCGATTCTCAGC |
| | TRB-V-H-1 | 0.1 μM | 5′ | GTAAAACGACGGCCAGTCTCAGGTCACCAGTTCCCTAAC |
| | TRB-V-I-1 | 0.1 μM | 5′ | GTAAAACGACGGCCAGTCCTAGATTTTCAGGTCGCCAGT |
| | TRB-V-Q-1 | 0.1 μM | 5′ | GTAAAACGACGGCCAGTCTCAACTAGACAAATCGGGGCT |
| | TRB-V-U-1 | 0.1 μM | 5′ | GTAAAACGACGGCCAGTATCGATTTTCTGCAGAGAGGCT |
| | TRB-V-Y-1 | 0.1 μM | 5′ | GTAAAACGACGGCCAGTCGGTATGCCCAACAATCGATTC |
| | TRB-V-AC-1 | 0.1 μM | 5′ | GTAAAACGACGGCCAGTCTGAAGGGTACAGCGTCTCTC |
| | TRB-V-AH-1 | 0.1 μM | 5′ | GTAAAACGACGGCCAGTTCCTCTGAGTCAACAGTCTCCA |
| | TRB-V-AK-1 | 0.1 μM | 5′ | GTAAAACGACGGCCAGTCTGAGGCCACATATGAGAGTGG |
| | TRB-J-L-1 | 0.1 μM | 3′ | TAATACGACTCACTATAGGGGAAAACTCACCCAGCACGGTC |
| | TRB-J-M-1 | 0.1 μM | 3′ | TAATACGACTCACTATAGGGTCACCCAGCACGGTCAGCC |
| | TRB-V-G-1 | 0.15 μM | 5′ | GTAAAACGACGGCCAGTGATTCTCAGGTCTCCAGTTCCC |
| | TRB-V-K-1 | 0.15 μM | 5′ | GTAAAACGACGGCCAGTTACCACTGGCAAAGGAGAAGTC |
| | TRB-V-T-1 | 0.15 μM | 5′ | GTAAAACGACGGCCAGTCAAAGGAGAAGTCTCAGATGGC |
| | TRB-V-AD-1 | 0.15 μM | 5′ | GTAAAACGACGGCCAGTTTTCTCATCAACCATGCAAGCC |
| | TRB-V-AF-1 | 0.15 μM | 5′ | GTAAAACGACGGCCAGTGGAGATGCACAAGAAGCGATTC |
| TRB D-J | TRB-J-A-1 | 0.025 μM | 3′ | TAATACGACTCACTATAGGGCTACAACTGTGAGTCTGGTGCC |
| | TRB-J-B-1 | 0.025 μM | 3′ | TAATACGACTCACTATAGGGCTACAACGGTTAACCTGGTCC |
| | TRB-J-C-1 | 0.025 μM | 3′ | TAATACGACTCACTATAGGGTACAACAGTGAGCCAACTTCCC |
| | TRB-J-D-1 | 0.025 μM | 3′ | TAATACGACTCACTATAGGGCAAGACAGAGAGCTGGGTTCC |
| | TRB-J-E-1 | 0.025 μM | 3′ | TAATACGACTCACTATAGGGCTAGGATGGAGAGTCGAGTCCC |
| | TRB-J-F-1 | 0.025 μM | 3′ | TAATACGACTCACTATAGGGCTGTCACAGTGAGCCTGGTC |
| | TRB-J-G-1 | 0.025 μM | 3′ | TAATACGACTCACTATAGGGCCTTCTTACCTAGCACGGTGAG |
| | TRB-J-H-1 | 0.025 μM | 3′ | TAATACGACTCACTATAGGGTTACCCAGTACGGTCAGCCTAG |
| | TRB-J-I-1 | 0.025 μM | 3′ | TAATACGACTCACTATAGGGCTTACCGAGCACTGTCAGCC |
| | TRB-J-J-1 | 0.025 μM | 3′ | TAATACGACTCACTATAGGGCTTACCCAGCACTGAGAGCC |
| | TRB-J-K-1 | 0.025 μM | 3′ | TAATACGACTCACTATAGGGTCACCGAGCACCAGGAGCC |
| | TRB-J-N-1 | 0.025 μM | 3′ | TAATACGACTCACTATAGGGGAATCTCACCTGTGACCGTGAG |
| | TRB-D-A-1 | 0.1 μM | 5′ | GTAAAACGACGGCCAGTCCTCCACTCCCCTCAAAGGA |
| | TRB-D-B-1 | 0.1 μM | 5′ | GTAAAACGACGGCCAGTCAGACTAACCTCTGCCACCTG |
| | TRB-J-L-1 | 0.1 μM | 3′ | TAATACGACTCACTATAGGGGAAAACTCACCCAGCACGGTC |
| | TRB-J-M-1 | 0.1 μM | 3′ | TAATACGACTCACTATAGGGTCACCCAGCACGGTCAGCC |
| TRG | TRG-V-E-1 | 0.05 μM | 5′ | GTAAAACGACGGCCAGTCAAGCATGAGGAGGAGCTGGAAATTG |
| | TRG-V-F-1 | 0.05 μM | 5′ | GTAAAACGACGGCCAGTACGTCTACATCCACTCTCACC |
| | TRG-V-A-1 | 0.1 μM | 5′ | GTAAAACGACGGCCAGTGCACAAGGAACAACTTGAGATTG |
| | TRG-V-B-1 | 0.1 μM | 5′ | GTAAAACGACGGCCAGTTGGAAGCACAAGGAAGAACTTGAGAA |
| | TRG-V-C-1 | 0.1 μM | 5′ | GTAAAACGACGGCCAGTGCACAGGGAAGAGCCTTAAATT |
| | TRG-V-D-1 | 0.1 μM | 5′ | GTAAAACGACGGCCAGTCAGGAGGTGGAGCTGGATATT |
| | TRG-V-G-1 | 0.1 μM | 5′ | GTAAAACGACGGCCAGTCTCTCACTTCAATCCTTACCATCAA |
| | TRG-V-H-1 | 0.2 μM | 5′ | GTAAAACGACGGCCAGTGCTCACACTTCCACTTCCACTTTGAAAATAAAGT |
| | TRG-J-A-1 | 0.2 μM | 3′ | TAATACGACTCACTATAGGGAGTGTTGTTCCACTGCCAAAG |
| | TRG-J-B-1 | 0.2 μM | 3′ | TAATACGACTCACTATAGGGGTTCCGGGACCAAATACCTTG |
| | TRG-J-C-1 | 0.2 μM | 3′ | TAATACGACTCACTATAGGGGAGCTTAGTCCCTTCAGCAAATA |
| | TRG-J-D-1 | 0.2 μM | 3′ | TAATACGACTCACTATAGGGCCTAGTCCCTTTTGCAAACG |
| TRD | TRD-V-A-1 | 0.2 μM | 5′ | GTAAAACGACGGCCAGTGAATGCAAAAAGTGGTCGCTATTC |
| | TRD-V-B-1 | 0.2 μM | 5′ | GTAAAACGACGGCCAGTTGCAAAGAACCTGGCTGTACT |
| | TRD-V-C-1 | 0.2 μM | 5′ | GTAAAACGACGGCCAGTTGCAGATTTTACTCAAGGACGG |
| | TRD-V-D-1 | 0.2 μM | 5′ | GTAAAACGACGGCCAGTGCAAAATGCAACAGAAGGTCG |
| | TRD-V-E-1 | 0.2 μM | 5′ | GTAAAACGACGGCCAGTGATAAAAATGAAGATGGAAGATTCACTGT |
| | TRD-V-F-1 | 0.2 μM | 5′ | GTAAAACGACGGCCAGTCTCCTTCAATAAAAGTGCCAAGC |
| | TRD-V-G-1 | 0.2 μM | 5′ | GTAAAACGACGGCCAGTATTGAAAAGAAGTCAGGAAGACTAAGT |
| | TRD-V-H-1 | 0.2 μM | 5′ | GTAAAACGACGGCCAGTTCCAGAAAGCAGCCAAATCC |
| | TRD-D-A-1 | 0.2 μM | 5′ | GTAAAACGACGGCCAGTAGGGGTATTGTGGATGGCAG |
| | TRD-J-A-1 | 0.2 μM | 3′ | TAATACGACTCACTATAGGGTTCCACAGTCACACGGGT |
| | TRD-J-B-1 | 0.2 μM | 3′ | TAATACGACTCACTATAGGGGGTTCCACGATGAGTTGTGTT |
| | TRD-J-C-1 | 0.2 μM | 3′ | TAATACGACTCACTATAGGGCACGAAGAGTTTGATGCCAGT |
| | TRD-J-D-1 | 0.2 μM | 3′ | TAATACGACTCACTATAGGGGTTGTTGTACCTCCAGATAGGTT |
| | TRD-J-E-1 | 0.2 μM | 3′ | TAATACGACTCACTATAGGGTGGCTAGAAACACTTACTTGCA |
| | TRD-D-B-1 | 0.2 μM | 3′ | TAATACGACTCACTATAGGGCCCAGGGAAATGGCACTTTTG |
| IGH D-J | IGH-D-A-1 | 0.2 μM | 5′ | GTAAAACGACGGCCAGTGATTCYGAACAGCCCCGAGTCA |
| | IGH-D-B-1 | 0.2 μM | 5′ | GTAAAACGACGGCCAGTGATTTTGTGGGGGYTCGTGTC |
| | IGH-D-C-1 | 0.2 μM | 5′ | GTAAAACGACGGCCAGTGTTTGRRGTGAGGTCTGTGTCA |
| | IGH-D-D-1 | 0.2 μM | 5′ | GTAAAACGACGGCCAGTGTTTRGRRTGAGGTCTGTGTCACT |
| | IGH-D-E-1 | 0.2 μM | 5′ | GTAAAACGACGGCCAGTCTTTTTGTGAAGGSCCCTCCTR |
| | IGH-D-F-1 | 0.2 μM | 5′ | GTAAAACGACGGCCAGTGTTATTGTCAGGSGRTGTCAGAC |
| | IGH-D-G-1 | 0.2 μM | 5′ | GTAAAACGACGGCCAGTGTTATTGTCAGGGGGTGYCAGRC |
| | IGH-D-H-1 | 0.2 μM | 5′ | GTAAAACGACGGCCAGTGTTTCTGAAGSTGTCTGTRTCAC |
| | IGH-J-A-1 | 0.4 μM | 3′ | TAATACGACTCACTATAGGGCTTACCTGAGGAGACGGTGACC |
| IGH V-J | IGH-V-FR1-B- | 0.1 μM | 5′ | GTAAAACGACGGCCAGTGCAGTCTGGAGCAGAGGTGAAAA |
| | 1 | | | |
| | IGH-V-FR1-E- | 0.1 μM | 5′ | GTAAAACGACGGCCAGTGAGGTGCAGCTGTTGGAGTC |
| | 1 | | | |
| | IGH-V-FR1-G- | 0.1 μM | 5′ | GTAAAACGACGGCCAGTCAGTGGGGCGCAGGACTGTT |
| | 1 | | | |
| | IGH-V-FR1-H- | 0.1 μM | 5′ | GTAAAACGACGGCCAGTCCAGGACTGGTGAAGCCTCC |
| | 1 | | | |
| | IGH-V-FR1-K- | 0.1 μM | 5′ | GTAAAACGACGGCCAGTCCTCAGTGAAGGTTTCCTGCAAGG |
| | 1 | | | |
| | IGH-V-FR1-L- | 0.1 μM | 5′ | GTAAAACGACGGCCAGTAAACCCACAGAGACCCTCACGCTGAC |
| | 1 | | | |
| | IGH-V-FR1-M- | 0.1 μM | 5′ | GTAAAACGACGGCCAGTCTGGGGGGTCCCTGAGACTCTCCTG |
| | 1 | | | |
| | IGH-V-FR1-N- | 0.1 μM | 5′ | GTAAAACGACGGCCAGTCTTCACAGACCCTGTCCCTCACCTG |
| | 1 | | | |
| | IGH-V-FR1-O- | 0.1 μM | 5′ | GTAAAACGACGGCCAGTTCGCAGACCCTCTCACTCACCTGTG |
| | 1 | | | |
| | IGH-J-A-1 | 0.1 μM | 3′ | TAATACGACTCACTATAGGGCTTACCTGAGGAGACGGTGACC |
| | IGH-J-B-1 | 0.1 μM | 3′ | TAATACGACTCACTATAGGGCTCACCTGAGGAGACGGTGACC |
| | IGH-V-FR1-A- | 0.2 μM | 5′ | GTAAAACGACGGCCAGTCTGGGGCTGAGGTGAAGAAG |
| | 1 | | | |
| | IGH-V-FR1-C- | 0.2 μM | 5′ | GTAAAACGACGGCCAGTTCACCTTGAAGGAGTCTGGTCC |
| | 1 | | | |
| | IGH-V-FR1-D- | 0.2 μM | 5′ | GTAAAACGACGGCCAGTAGGTGCAGCTGGTGGAGTC |
| | 1 | | | |
| | IGH-V-FR1-F- | 0.2 μM | 5′ | GTAAAACGACGGCCAGTCCAGGACTGGTGAAGCCTTC |
| | 1 | | | |
| | IGH-V-FR1-I- | 0.2 μM | 5′ | GTAAAACGACGGCCAGTGTACAGCTGCAGCAGTCAGG |
| | 1 | | | |
| | IGH-V-FR1-J- | 0.2 μM | 5′ | GTAAAACGACGGCCAGTGCTGGTGCAATCTGGGTCTG |
| | 1 | | | |
| IGK-A | IGK-V-A-1 | 0.1 μM | 5′ | GTAAAACGACGGCCAGTAAGTGGGGTCCCATCAAGGTTCAG |
| | IGK-V-B-1 | 0.1 μM | 5′ | GTAAAACGACGGCCAGTAGTCCCATCTCGGTTCAGTGGCAG |
| | IGK-V-C-1 | 0.1 μM | 5′ | GTAAAACGACGGCCAGTGAAACAGGGGTCCCATCAAGGTTC |
| | IGK-V-D-1 | 0.1 μM | 5′ | GTAAAACGACGGCCAGTTCCCAGACAGATTCAGTGGCAGTG |
| | IGK-V-E-1 | 0.1 μM | 5′ | GTAAAACGACGGCCAGTCTGGAGTGCCAGATAGGTTCAGTG |
| | IGK-V-F-1 | 0.1 μM | 5′ | GTAAAACGACGGCCAGTCCCTGGAGTCCCAGACAGGTTCAG |
| | IGK-V-G-1 | 0.1 μM | 5′ | GTAAAACGACGGCCAGTGCATCCCAGCCAGGTTCAGTG |
| | IGK-V-H-1 | 0.1 μM | 5′ | GTAAAACGACGGCCAGTGTCCCTGACCGATTCAGTGGCA |
| | IGK-V-I-1 | 0.1 μM | 5′ | GTAAAACGACGGCCAGTAATCCCACCTCGATTCAGTGGC |
| | IGK-V-J-1 | 0.1 μM | 5′ | GTAAAACGACGGCCAGTCTCAGGGGTCCCCTCGAGGTT |
| | IGK-V-K-1 | 0.1 μM | 5′ | GTAAAACGACGGCCAGTAGACACTGGGGTCCCAGCCA |
| | IGK-DE-A-1 | 0.1 μM | 3′ | TAATACGACTCACTATAGGGGCAGCTGCAGACTCATGAGGAG |
| | IGk-J-A-1 | 0.1 μM | 3′ | TAATACGACTCACTATAGGGACGTTTGATCTCCACCTTGGTCCC |
| | IGK-J-B-1 | 0.1 μM | 3′ | TAATACGACTCACTATAGGGACGTTTGATATCCACTTTGGTCCC |
| | IGK-J-C-1 | 0.1 μM | 3′ | TAATACGACTCACTATAGGGACGTTTAATCTCCAGTCGTGTCCC |
| IGK-B | IGK-INTR-A-1 | 0.1 μM | 5′ | GTAAAACGACGGCCAGTGAGTGGCTTTGGTGGCCATGC |
| | IGK-DE-A-1 | 0.1 μM | 3′ | TAATACGACTCACTATAGGGCAGCTGCAGACTCATGAGGAG |
| 2^{nd} | | | | |
| step | Primer | | Primer | Barcoded primer sequence with M13 adapter |
| PCR | nomenclature | μM in PCR | direction | (forward/reverse) 5′ to 3′ |
| forward | Ill-D501-F | 0.2 μM | 5′ | AATGATACGGCGACCACCGAGATCTACACTATAGCCTACACTCTTTCCCTACACGACGCTCTTCCGATCTGTAAAACGACGGCCAGT |
| | Ill-D502-F | 0.2 μM | 5′ | AATGATACGGCGACCACCGAGATCTACACATAGAGGCACACTCTTTCCCTACACGACGCTCTTCCGATCTGTAAAACGACGGCCAGT |
| | Ill-D503-F | 0.2 μM | 5′ | AATGATACGGCGACCACCGAGATCTACACCCTATCCTACACTCTTTCCCTACACGACGCTCTTCCGATCTGTAAAACGACGGCCAGT |
| | Ill-D504-F | 0.2 μM | 5′ | AATGATACGGCGACCACCGAGATCTACACGGCTCTGAACACTCTTTCCCTACACGACGCTCTTCCGATCTGTAAAACGACGGCCAGT |
| | Ill-D505-F | 0.2 μM | 5′ | AATGATACGGCGACCACCGAGATCTACACAGGCGAAGACACTCTTTCCCTACACGACGCTCTTCCGATCTGTAAAACGACGGCCAGT |
| | Ill-D506-F | 0.2 μM | 5′ | AATGATACGGCGACCACCGAGATCTACACTAATCTTAACACTCTTTCCCTACACGACGCTCTTCCGATCTGTAAAACGACGGCCAGT |
| | Ill-D507-F | 0.2 μM | 5′ | AATGATACGGCGACCACCGAGATCTACACCAGGACGTACACTCTTTCCCTACACGACGCTCTTCCGATCTGTAAAACGACGGCCAGT |
| | Ill-D508-F | 0.2 μM | 5′ | AATGATACGGCGACCACCGAGATCTACACGTACTGACACACTCTTTCCCTACACGACGCTCTTCCGATCTGTAAAACGACGGCCAGT |
| reverse | Ill-D701-R | 0.2 μM | 3′ | CAAGCAGAAGACGGCATACGAGATCGAGTAATGTGACTGGAGTTCAGACGTGTGCTCTTCCGATCTTAATACGACTCACTATAGGG |
| | Ill-D702-R | 0.2 μM | 3 | CAAGCAGAAGACGGCATACGAGATTCTCCGGAGTGACTGGAGTTCAGACGTGTGCTCTTCCGATCTTAATACGACTCACTATAGGG |
| | Ill-D703-R | 0.2 μM | 3′ | CAAGCAGAAGACGGCATACGAGATAATGAGCGGTGACTGGAGTTCAGACGTGTGCTCTTCCGATCTTAATACGACTCACTATAGGG |
| | Ill-D704-R | 0.2 μM | 3′ | CAAGCAGAAGACGGCATACGAGATGGAATCTCGTGACTGGAGTTCAGACGTGTGCTCTTCCGATCTTAATACGACTCACTATAGGG |
| | Ill-D705-R | 0.2 μM | 3′ | CAAGCAGAAGACGGCATACGAGATTTCTGAATGTGACTGGAGTTCAGACGTGTGCTCTTCCGATCTTAATACGACTCACTATAGGG |
| | Ill-D706-R | 0.2 μM | 3′ | CAAGCAGAAGACGGCATACGAGATACGAATTCGTGACTGGAGTTCAGACGTGTGCTCTTCCGATCTTAATACGACTCACTATAGGG |
| | Ill-D707-R | 0.2 μM | 3′ | CAAGCAGAAGACGGCATACGAGATAGCTTCAGGTGACTGGAGTTCAGACGTGTGCTCTTCCGATCTTAATACGACTCACTATAGGG |
| | Ill-D708-R | 0.2 μM | 3′ | CAAGCAGAAGACGGCATACGAGATGCGCATTAGTGACTGGAGTTCAGACGTGTGCTCTTCCGATCTTAATACGACTCACTATAGGG |
| | Ill-D709-R | 0.2 μM | 3′ | CAAGCAGAAGACGGCATACGAGATCATAGCCGGTGACTGGAGTTCAGACGTGTGCTCTTCCGATCTTAATACGACTCACTATAGGG |
| | Ill-D710-R | 0.2 μM | 3′ | CAAGCAGAAGACGGCATACGAGATTTCGCGGAGTGACTGGAGTTCAGACGTGTGCTCTTCCGATCTTAATACGACTCACTATAGGG |
| | Ill-D711-R | 0.2 μM | 3′ | CAAGCAGAAGACGGCATACGAGATGCGCGAGAGTGACTGGAGTTCAGACGTGTGCTCTTCCGATCTTAATACGACTCACTATAGGG |
| | Ill-D712-R | 0.2 μM | 3′ | CAAGCAGAAGACGGCATACGAGATCTATCGCTGTGACTGGAGTTCAGACGTGTGCTCTTCCGATCTTAATACGACTCACTATAGGG |

Primer3³¹, Primer Digital (PrimerDigital Ltd, Helsinki, Finland) MFEprimer-2.0³² and Oligo (Molecular Biology Insights, Inc., Colorado, USA) were used for checking primer specificity and multiplexing. Primer design criteria were followed for all loci: primer melting temperature 57-63° C.; comparable size of final amplicon; primer length 20-24 nt; avoidance of primer dimers; minimal distance of 3′primer end to the junctional region of, preferably, >10-15 bp to avoid false negativity for rearrangements with larger nucleotide deletions from the germline sequence; avoidance of regions with known single nucleotide polymorphisms to allow identical primer annealing for all alleles of the respective V, D or J genes; targeting of, preferably, all V, D and J genes known to be rearranged plus the intronRSS and Kde regions for IGK.

Following in silico design, primers were first tested in monoplex and multiplex reactions using primary patient samples or cell lines with defined rearrangements. In occasional cases where no such samples were available, healthy tonsil or mononuclear DNA samples were employed. Oligoclonal template pools were then created from mixtures of rearranged cell lines and diagnostic samples with defined rearrangements covering many different V, D and/or J genes. Alternatively, for some loci, plasmid pools were produced, covering as many different rearrangements as possible. These multi-target pools allowed fine-tuning of reaction conditions and/or primer concentrations to assess comparable amplification efficiencies. This iterative process of testing also led to a reduction of primers if these appeared redundant. Further multicentre testing was performed with a limited number of monoclonal and poly/oligoclonal samples and on different sequencing platforms, which allowed assessment of robustness of the primer mixes and protocols.

Since the assays were designed with the aim to be platform-independent, a two-step PCR was employed, enabling to switch the sequencing adaptors and to reduce the total number of primers even if a large number of barcodes is necessary. Also, maximal amplicon lengths were defined with respect to the possible maximal sequencing read lengths of current sequencers. PCR conditions were optimized with the aim to find optimal conditions common for all reactions, thus allowing for parallel library preparation. Various numbers of PCR cycles in 1^{st} and 2^{nd} PCR, different polymerases and several library purification methods were tested and compared.

Although this study was exclusively performed on the Illumina MiSeq, the applicability of the same PCR panel on the IonTorrent instrument (ThermoFischer Scientific) was tested in a single-centre setting and a one-step Illumina MiSeq PCR approach was also tested in a single-center setting.

### Multicentre Validation of Assays for MRD Marker Identification in ALL

Five experienced EuroClonality-NGS laboratories tested the robustness and applicability of the optimized assays for IG/TR marker identification in ALL in comparison to standard techniques. All laboratories (Bristol/London, Paris, Monza, Prague and Kiel) are members of the EuroMRD consortium and reference laboratories for ALL MRD analysis. Each of the participating laboratories performed NGS-based IG/TR MRD marker identification in 10 patients with B- or T-lineage ALL. A central standard operating procedure was strictly followed by all laboratories. The study was executed using the Illumina MiSeq (2×250 bp v2 kit). NGS analyses were performed fully in parallel to conventional PCR plus Sanger sequencing of clonal products following standard guidelines¹¹. For a part of the cases with unexplained discrepant results between the two methods, allele-specific PCR assays (either for digital droplet PCR or real-time quantitative PCR) were designed to clarify if the respective clonal rearrangement represented the leukemic bulk. EuroMRD guidelines were used to design and interpret allele-specific PCR assays^{33,34}.

### Results

### Primer Design and Technical Validation of Primer Performance

Based on the results of the testing and validation phases, the final IG/TR primer mixes consist of eight tubes with 92 forward and 30 reverse primers, 15 of the latter being used in pairs of different tubes). Primer positions and sequences are presented in FIG. 5 and Table 3.

### Implementation of Quality Control Compositions

Quality control of robust amplification, library preparation and sequencing are of utmost importance for these complex assays. Different primers need to work under the same reaction conditions, while additional variability can be introduced by sample characteristics and sequencing. Primer performance has to be monitored longitudinally, and for the exact estimation of clonal abundance it is important to correct for the number of sequencing reads per input molecule.

To address these issues, two types of quality control compositions were included: (i) the cIT-QC of Example 1 was spiked to each tube as library control and calibrator, and (ii) the cPT-QC of Example 2 was run in parallel to monitor general primer performance and sequencing.

### Laboratory Protocol

Primers were tailed with universal and T7-linker sequences, and divided over eight tubes (IGH-VJ, IGH-DJ, IGK-VJ-Kde, intron-Kde, TRB-VJ, TRB-DJ, TRG, TRD). The PCR protocol is summarized in Table 4. Sequencing libraries were prepared via a two-step PCR, each using a final reaction volume of 50 μl with 100 ng diagnostic DNA and 10 ng of polyclonal DNA. For the cIT-QC, genomic DNA of 40 cell equivalents of each the 9 different cell lines were spiked into all samples. MgCl₂ was intended to be used at a final concentration of 1.5 mM, but needed optimization for some tubes. Therefore, master-mixes for the 1^{st} PCR were tube-specific, but the temperature profile was uniform for all tubes.

After the 1^{st} round of PCR, gel electrophoresis was performed to check for the successful amplification of all targets. For TRB, gel extraction of the specific PCR products was performed prior to the 2^{nd} PCR.

All first round PCR products, except for TRB, the PCR products were diluted 1:50 unless amplicons were very weak. The TRB PCR products and PCR products with weak amplicons were used undiluted. Master-mixes for the 2^{nd} PCR and the temperature profiles were identical for all tubes (Table 4). Primers for the 2^{nd} PCR contained sequencing adaptors and sequencing indexes (barcodes). Unique combination of forward and reverse indexes was used for each library. Three μl of undiluted TRB PCR products and 1 μl of 1:50-diluted IGH, IGK, TRG, and TRD PCR products were amplified in the 2^{nd} PCR.

TABLE 4
Standardized PCR protocol. (A) Reaction conditions of 1^{st} and 2^{nd} PCR. (B) PCR Cycling conditions.
A
1^{st} PCR

| | | | | | | | | | IGK-VJ-Kde, | | | | | | | | | | | |
|---|---|---|---|---|---|---|---|---|---|---|---|---|---|---|---|---|---|---|---|---|
| | | | IGH-VJ | | | IGH-DJ | | | Intron-Kde | | | TRS-VJ, TRB-DJ | | | TRG | | | TRD | | |
| | Stock | | Final | | | Final | | | Final | | | Final | | | Final | | | Final | | |
| | concen- | | concen- | | | concen- | | | concen- | | | concen- | | | concen- | | | concen- | | |
| | tration | | tration | | [symbol] | tration | | [symbol] | tration | | [symbol] | tration | | [symbol] | tration | | [symbol] | tration | | [symbol] |
| PCR Buffer [symbol] | 10x | | 1x | | 5 | 1x | | 5 | 1x | | 5 | 1x | | 5 | 1x | | 5 | 1x | | 5 |
| MgCl2 | 25 | mM | 2.5 | mM | 5 | 3 | mM | 6 | 1.5 | mM | 3 | 4 | mM | 8 | 4 | mM | 8 | 2 | mM | 4 |
| dNTP-Mix | 10 | mM | 0.2 | mM | 1 | 0.4 | mM | 2.0 | 0.2 | mM | 1 | 0.2 | mM | 1 | 0.2 | mM | 1 | 0.2 | mM | 1 |

| | | | | | | | | | | | | | |
|---|---|---|---|---|---|---|---|---|---|---|---|---|---|
| EagleTaq/AmpliTaq | 5 [symbol] | 1 [symbol] | 0.2 | 1.5 [symbol] | 0.3 | 1 [symbol] | 0.2 | 1 [symbol] | 0.2 | 1 [symbol] | 0.2 | 1 [symbol] | 0.8 |
| Gold | | | | | | | | | | | | | |

Reaction volume: 50 μl

2^{nd} PCR

[symbol]

| | Stock | Final | |
|---|---|---|---|
| | concentration | concentration | μl/sample |
| PCR Buffer with | 10x | 1x | 5 |
| MgCl2 | 18 mM | 1.8 mM | 0 |
| dNTP-Mix | 10 mM | 0.2 mM | 1 |
| Fast Start High | 5 [symbol] | 2.5 [symbol] | 0.5 |
| Fidelity polymerase | | | |

Reaction volume: 50 μl

B

| 1^{st} PCR | | | | | 2^{nd} PCR | | | | |
|---|---|---|---|---|---|---|---|---|---|
| | initial | | | | | initial | | | |
| | denaturation | 94° C. | 10 min | | | denaturation | 95° C. | 2 min | |
| 35 | denaturation | 94° C. | 1 | min | 20 | denaturation | 94° C. | 30 | sec |
| cycles | annealing | 63° C. | 1 | min | cycles | annealing | 63° C. | 30 | sec |
| | extension | 72° C. | 30 | sec | | extension | 72° C. | 30 | sec |
| | final | 72° C. | 30 | min | | final | 72° C. | 5 | min |
| | extension | | | | | extension | | | |

| | | | |
|---|---|---|---|
| 12° C. | — | 12° C. | — |

[symbol]

indicates data missing or illegible when filed

Following 2^{nd} PCR, products from all samples of a run were pooled in equimolar ratios into 8 tube-wise subpools and purified by gel-extraction (see Table 5 for the amplicon lengths). Finally, the subpools were pooled equimolarly into one final pool. Sequencing was performed on Illumina MiSeq sequencers, using 2×250 bp v2 chemistry with a final concentration of 7 pM for the amplicon library and 10% PhiX control added to avoid low-complexity library issues.

TABLE 5
Mean size of PCR products after the 2^{nd} PCR (containing
the Illumina sequencing adaptors and barcodes).

| | Amplicon length |
|---|---|
| Gene | (bp) |
| TRB-VJ | 309-407 |
| TRB-DJ | 300-408 |
| TRG | 256-360 |
| TRD | 309-450 |
| IGH-VJ | 484-681 |
| IGH-DJ | 266-358 |
| IGK-VJ-Kde | 296-384 |
| intron-Kde | 309-382 |

### Bioinformatic Protocol

ARResT/Interrogate was the main bioinformatics platform used in this study, along with Vidjil⁴⁷ and IMGT⁴⁸ resources for specific aspects of this work. Demultiplexing was performed accepting no mismatches. Reads were annotated with EuroClonality-NGS primer sequences (to trim non-amplicon sequence, and for the cPT-QC-based quality control), paired-end joined, dereplicated, immunogenetically annotated⁴⁸, and classified into rearrangement types (complete and incomplete, and other special types like intron-Kde rearrangements), or “junction classes”. Reads with no rearrangement were excluded from the total read count used for relative abundances.

cIT-QC sequences described above were identified in the data through their immunogenetic annotation. Their counts served both as ‘in-tube’ control and for normalization per primer set: total cIT-QC cells are divided by cIT-QC total reads, the resulting factor used to convert rearrangement reads to cells, those cells divided by total input cells (15,000 in this example). Identified IG/TR sequences were defined as index sequences if abundance after cIT-QC normalisation exceeded 5%. ARResT/Interrogate can track the DNJ 3′stem of a junction, the sequence remaining stable during IGH or TRB clonal evolution in case of V-replacement or ongoing V to DJ rearrangements. The stem consists of the last ⋅3 nt of D (or of the NDN if no D is identifiable), any and all of N2 nucleotides, and the J nucleotides of the junction. This stem is available as a separate immunogenetic feature across all samples and thus able to link other features, e.g. clonotypes.

### Multicentre Validation of Assays for MRD Marker Identification in ALL

Next, fifty ALL diagnostic samples (29 BCP-ALL and 21 T-ALL) were analysed for the multicentre validation study. Each of the five participating laboratories received preconfigured 96-well plates containing the different multiplexed NGS primer combinations per target (FIG. 4).

In summary, 96 libraries were generated per lab (total of 480 libraries), and sequenced with a total output of 47M reads (⋅9.2M/lab). Centralised analysis was performed with ARResT/Interrogate⁴³ using IMGT germline sequences⁴⁸—further analyses and verifications were performed with Vidjil⁴⁷ and IMGT/V-QUEST⁴⁸.

Overall, 311 clonal IG/TR rearrangements (clonotypes) were identified, with a mean of 5.9 (0-14)/sample by NGS (a 5% threshold was applied for NGS after cIT-QC-based normalization) vs. 5.0 (0-14)/sample by Sanger, while 217 (45%) libraries demonstrated no clonotypes above threshold by either method. A total of 196/311 (63%) clonotypes were fully concordant between NGS and Sanger (FIG. 6). NGS exclusively identified 63 index sequences, whereas 52 IG/TR Sanger sequences were not assigned as NGS index sequence by ARResT/Interrogate. 26 NGS pos/Sanger neg cases showed a clonal PCR product also in the respective low-throughput approach but subsequent Sanger sequencing failed due to polyclonal background, mixed sequences or weak PCR products. In an additional 6 Sanger neg/NGS pos cases, the respective primer was missing in the low-throughput approach. For the remaining 31 discrepancies no technical explanation for Sanger failure could be found (in 16/19 q/ddPCR evaluated cases the rearrangement was confirmed by ASO-PCR, in 3/16 on a subclonal level).

Conversely, 52 clonal IG/TR rearrangements were only detected by Sanger when the 5% NGS threshold was applied: for 5 sequences (1 TRG, 2 TRB-VJ, and 2 IGH-DJ) the relevant primer was not present in the NGS primer set, in 12 cases no explanation was found for the discrepancy. However, in the majority of discordant cases (35/52) the Sanger identified sequences (7 TRD, 8 TRB-VJ, 6 TRG, 4 TRB-DJ, 2 IGK-VJ-Kde, 5 IGH-VJ, 3 IGH-DJ) were also detectable by NGS, but with and abundance below 5%. In 36/39 q/ddPCR evaluated cases the rearrangement was confirmed by ASO-PCR, including all low NGS positive sequences, in 14/36 cases on a subclonal level. Overall concordance between Sanger and NGS, including negative libraries, was 78%.

In 12/29 B-lineage ALL samples the evolution of the dominant clonal IGH sequence was identified employing ARResT/Interrogate. The evolved clonotypes shared the DNJ stem with the dominant one, but the VND part of the rearrangement differed.

The assay performance was also analysed by standardized evaluation of QC samples (cIT-QC and cPT-QC). This showed a remarkably high intra- and inter-lab consistency without statistically significant differences between the five labs.

### Suitable Modifications of the Central SOP for MRD Marker Identification

During the process of multicentre validation, suitable modifications of the SOP were tested in particular laboratories as parallel actions.

One-step versus two-step PCR: The EuroClonality-NGS working group decided to use two-step PCR to enable switching of sequencing adaptors and to limit the total number of required primer batches even if a large number of barcodes is necessary. As first round PCR products are not barcoded, identification of contamination phenomena is hampered in this approach. Therefore, a one-step PCR was tested in a single center (Paris) as an alternative for laboratories that are able to maintain higher numbers of different primer batches. The one-step approach reduces the risk of contamination and thus favours use of NGS not only for marker identification, but also for MRD assessment. The standard operating procedures are shown in supplementary information.

Bead extraction: In our single target evaluation and validation phase, gel extraction of the specific TRB amplicons turned out to lead to more specific libraries compared to bead extraction. However, gel extraction is not used in all laboratories, therefore, in a later phase of the study bead purification of all libraries was also tested. Optimization of the purification processes led to comparable ratios of specific reads irrespective of the type of library purification.

Withdrawal of addition of polyclonal DNA to reaction mix: Polyclonal DNA was added to each reaction in order to prevent excessive primer dimer formation in samples lacking particular rearrangements. The addition of polyclonal DNA, however, alters the composition of polyclonal background of the samples and hampers the analysis of the immune repertoire. We therefore performed testing on 4 samples with B and 4 samples with T cell aplasia and showed that addition of cIT-QC is sufficient to prevent the excessive formation of unspecific PCR products.

### REFERENCES

1 Tonegawa S. Somatic generation of antibody diversity. Nature 1983; 302: 575-581.
2 Davis M M, Bjorkman P J. T-cell antigen receptor genes and T-cell recognition. Nature 1988; 334: 395-402.
3 Schlissel M S. Regulating antigen-receptor gene assembly. Nat Rev Immunol 2003; 3: 890-899.
4 Lefranc M-P, Lefranc G. The T cell receptor factsbook. Academic Press, 2001.
5 Lefranc M-P, Lefranc G. The immunoglobulin factsbook. Academic Press, 2001.
6 Monroe J G, Dorshkind K. Fate Decisions Regulating Bone Marrow and Peripheral B Lymphocyte Development. In: Advances in immunology. 2007, pp 1-50.
7 von Boehmer H, Melchers F. Checkpoints in lymphocyte development and autoimmune disease. Nat Immunol 2010; 11: 14-20.
8 Evans P A S, Pott C, Groenen P J T A, Salles G, Davi F, Berger F et al. Significantly improved PCR-based clonality testing in B-cell malignancies by use of multiple immunoglobulin gene targets. Report of the BIOMED-2 Concerted Action BHM4-CT98-3936. Leukemia 2007; 21: 207-214.
9 Brüggemann M, White H, Gaulard P, Garcia-Sanz R, Gameiro P, Oeschger S et al. Powerful strategy for polymerase chain reaction-based clonality assessment in T-cell malignancies Report of the BIOMED-2 Concerted Action BHM4 CT98-3936. Leukemia 2007; 21: 215-221.
10 Langerak A W, Groenen P J T A, Brüggemann M, Beldjord K, Bellan C, Bonello L et al. EuroClonality/BIOMED-2 guidelines for interpretation and reporting of Ig/TCR clonality testing in suspected lymphoproliferations. Leukemia 2012; 26: 2159-2171.
11 van Dongen J J M, Langerak A W, Brüggemann M, Evans P A, Hummel M, Lavender F L et al. Design and standardization of PCR primers and protocols for detection of clonal immunoglobulin and T-cell receptor gene recombinations in suspect lymphoproliferations: report of the BIOMED-2 Concerted Action BMH4-CT98-3936. Leukemia 2003; 17: 2257-2317.
12 Boyd S D, Marshall E L, Merker J D, Maniar J M, Zhang L N, Sahaf B et al. Measurement and clinical monitoring of human lymphocyte clonality by massively parallel VDJ pyrosequencing. Sci Transl Med 2009; 1: 12ra23.
13 DeKosky B J, Ippolito G C, Deschner R P, Lavinder J J, Wine Y, Rawlings B M et al. High-throughput sequencing of the paired human immunoglobulin heavy and light chain repertoire. Nat Biotechnol 2013; 31: 166-169.
14 Freeman J D, Warren R L, Webb J R, Nelson B H, Holt R A. Profiling the T-cell receptor beta-chain repertoire by massively parallel sequencing. Genome Res 2009; 19: 1817-1824.
15 Gawad C, Pepin F, Carlton V E H, Klinger M, Logan A C, David B et al. Massive evolution of the immunoglobulin heavy chain locus in children with B precursor acute lymphoblastic leukemia Massive evolution of the immunoglobulin heavy chain locus in children with B precursor acute lymphoblastic leukemia. 2012; 120: 4407-4417.
16 Logan A C, Gao H, Wang C, Sahaf B, Jones C D, Marshall E L et al. High-throughput VDJ sequencing for quantification of minimal residual disease in chronic lymphocytic leukemia and immune reconstitution assessment. Proc Natl Acad Sci USA 2011; 108: 21194-21199.
17 Logan A C, Zhang B, Narasimhan B, Carlton V, Zheng J, Moorhead M et al. Minimal residual disease quantification using consensus primers and high-throughput IGH sequencing predicts post-transplant relapse in chronic lymphocytic leukemia. Leukemia 2013; 27: 1659-1665.
18 Robins H S, Srivastava S K, Campregher P V, Turtle C J, Andriesen J, Riddell S R et al. Overlap and Effective Size of the Human CD8+ T Cell Receptor Repertoire. Sci Transl Med 2010; 2: 47ra64-47ra64.
19 Wang C, Sanders C M, Yang Q, Schroeder H W, Wang E, Babrzadeh F et al. High throughput sequencing reveals a complex pattern of dynamic interrelationships among human T cell subsets. Proc Natl Acad Sci 2010; 107: 1518-1523.
20 Wu D, Sherwood A, Fromm J R, Winter S S, Dunsmore K P, Loh M L et al. High-throughput sequencing detects minimal residual disease in acute T lymphoblastic leukemia. Sci Transl Med 2012; 4: 134ra63.
21 Wu Y-C, Kipling D, Leong H S, Martin V, Ademokun A A, Dunn-Walters D K. High-throughput immunoglobulin repertoire analysis distinguishes between human IgM memory and switched memory B-cell populations. Blood 2010; 116: 1070-1078.
22 Bartram J, Goulden N, Wright G, Adams S, Brooks T, Edwards D et al. High throughput sequencing in acute lymphoblastic leukemia reveals clonal architecture of central nervous system and bone marrow compartments. Haematologica 2018; 103: e110-e114.
23 Faham M, Zheng J, Moorhead M, Carlton V E, Stow P, Coustan-Smith E et al. Deep-sequencing approach for minimal residual disease detection in acute lymphoblastic leukemia. Blood 2012; 120: 5173-5180.
24 Ladetto M, Bruggemann M, Monitillo L, Ferrero S, Pepin F, Drandi D et al. Next-generation sequencing and real-time quantitative PCR for minimal residual disease detection in B-cell disorders. Leukemia 2014; 28: 1299-1307.
25 Pulsipher M A, Carlson C, Langholz B, Wall D A, Schultz K R, Bunin N et al. IgH-V(D)J NGS-MRD measurement pre- and early post-allo-transplant defines very low and very high risk ALL patients. Blood 2015; 125: 3501-3508.
26 Kotrova M, Muzikova K, Mejstrikova E, Novakova M, Bakardjieva-Mihaylova V, Fiser K et al. The predictive strength of next-generation sequencing MRD detection for relapse compared with current methods in childhood ALL. Blood 2015; 126: 1045-7.
27 Langerak A W, Brüggemann M, Davi F, Darzentas N, Gonzalez D, Cazzaniga G et al. High throughput immunogenetics for clinical and research applications in immunohematology: potential and challenges. J Immunol 2017; 198: 3765-3774.
28 Kotrova M, van der Velden V H J, van Dongen J J M, Formankova R, Sedlacek P, Brüggemann M et al. Next-generation sequencing indicates false-positive MRD results and better predicts prognosis after SCT in patients with childhood ALL. Bone Marrow Transplant 2017; 52: 962-968.
29 Kotrova M, Trka J, Kneba M, Brüggemann M. Is Next-Generation Sequencing the way to go for Residual Disease Monitoring in Acute Lymphoblastic Leukemia? Mol Diagn Ther 2017. doi:10.1007/s40291-017-0277-9.
30 Pott C. Minimal Residual Disease Detection in Mantle Cell Lymphoma: Technical Aspects and Clinical Relevance. Semin Hematol 2011; 48: 172-184.
31 Ferrero S, Drandi D, Mantoan B, Ghione P, Omedè P, Ladetto M. Minimal residual disease detection in lymphoma and multiple myeloma: Impact on therapeutic paradigms. Hematol. Oncol. 2011; 29: 167-176.
32 Brüggemann M, Gökbuget N, Kneba M. Acute Lymphoblastic Leukemia: Monitoring Minimal Residual Disease as a Therapeutic Principle. Semin Oncol 2012; 39: 47-57.
33 Brüggemann M, Raff T, Kneba M. Has MRD monitoring superseded other prognostic factors in adult ALL? Blood 2012; 120: 4470-4481.
34 van Dongen J J M, Seriu T, Panzer-Grumayer E R, Biondi A, Pongers-Willemse M J, Corral L et al. Prognostic value of minimal residual disease in acute lymphoblastic leukaemia in childhood. Lancet 1998; 352: 1731-1738.
35 Brüggemann M, Kotrova M. Minimal residual disease in adult ALL: technical aspects and implications for correct clinical interpretation. Hematol Am Soc Hematol Educ Progr 2017: 13-21.
36 Logan A C, Vashi N, Faham M, Carlton V, Kong K, Buño I et al. Immunoglobulin and t cell receptor gene high-throughput sequencing quantifies minimal residual disease in acute lymphoblastic leukemia and predicts post-transplantation relapse and survival. Biol Blood Marrow Transplant 2014; 20: 1307-1313.
37 Wren D, Walker B A, Brüggemann M, Catherwood M A, Pott C, Stamatopoulos K et al. Comprehensive translocation and clonality detection in lymphoproliferative disorders by next-generation sequencing. Haematologica. 2017; 102: e57-e60.
38 Hardwick S A, Deveson I W, Mercer T R. Reference standards for next-generation sequencing. Nat. Rev. Genet. 2017; 18: 473-484.
39 Gargis A S, Kalman L, Lubin I M. Assuring the quality of next-generation sequencing in clinical microbiology and public health laboratories. J Clin Microbiol 2016; 54: 2857-2865.
Endrullat C, Glökler J, Franke P, Frohme M. Standardization and quality management in next-generation sequencing. Appl. Transl. Genomics. 2016; 10: 2-9.
41 Kurtz D M, Green M R, Bratman S V., Scherer F, Liu C L, Kunder C A et al. Noninvasive monitoring of diffuse large B-cell lymphoma by immunoglobulin high-throughput sequencing. Blood 2015; 125: 3679-3687.
42 Pulsipher M A, Carlson C, Langholz B, Wall D A, Schultz K R, Bunin N et al. IgH-V(D) J NGS-MRD measurement pre- and early post-allotransplant defines very low- and very high-risk ALL patients. Blood 2015; 125: 3501-3509.
43 Bystry V, Reigl T, Krejci A, Demko M, Hanakova B, Grioni A et al. ARResT/Interrogate: an interactive immunoprofiler for IG/TR NGS data. Bioinformatics 2016; 33: btw634.
44 Grupp S A, Kalos M, Barrett D, Aplenc R, Porter D L, Rheingold S R et al. Chimeric Antigen Receptor-Modified T Cells for Acute Lymphoid Leukemia. N Engl J Med 2013; 368:1509-1518.
45 Tang M, Wang G, Kong S K, Ho HP4. A Review of Biomedical Centrifugal Microfluidic Platforms. Micromachines (Basel) 2016; 7: E26.
46 Langerak A W, Szczepa⋅ski T, Van Der Burg M, Wolvers-Tettero I L M, Van Dongen J J M. Heteroduplex PCR analysis of rearranged T cell receptor genes for clonality assessment in suspect T cell proliferations. Leukemia 1997; 11: 2192-2199.
47 Duez M, Giraud M, Herbert R, Rocher T, Salson M, Thonier F. Vidjil: A Web Platform for Analysis of High-Throughput Repertoire Sequencing. PLoS One 2016; 11: e0166126.
48 Lefranc M P, Giudicelli V, Duroux P, Jabado-Michaloud J, Folch G, Aouinti S, Carillon E, Duvergey H, Houles A, Paysan-Lafosse T, Hadi-Saljoqi S, Sasorith S, Lefranc G, Kossida S. IMGT®, the international ImMunoGeneTics information System® 25 years on. Nucleic Acids Res 2015; 43: D413-22.

1. A composition comprising a mixture of genomic DNA isolated from a set of nine cultured cell lines, said set comprising the B cell lines ALL/MIK (B cell precursor ALL), Raji (Burkitt lymphoma), REH (B cell precursor ALL), TMM (CML-BC/EBV+B-LCL), TOM-1 (B cell precursor ALL), WSU-NHL (B cell lymphoma) and the T cell lines JB6 (ALCL), Karpas299 (ALCL) and MOLT-13 (T-ALL), or wherein one or more cell lines of said set is replaced with one or more other cell line(s) comprising the same immunoglobulin (IG)/T cell receptor (TR) gene rearrangements.

2. The composition according to claim 1, comprising a mixture of genomic DNA isolated from the B cell lines ALL/MIK, Raji, REH, TMM, TOM-1, WSU-NHL and the T cell lines JB6, Karpas299 and MOLT-13.

3. The composition according to claim 1, wherein said composition comprises essentially equal amounts of genomic DNA of each of said cell lines.

4. A composition consisting of essentially equimolar amounts of genomic DNA isolated from healthy human thymus, healthy human tonsil and healthy human peripheral blood mononuclear cells.

5. The composition according to claim 4, wherein, for each tissue, the genomic DNA is obtained from a number, preferably 3 to 10, different human individuals.

6. A diagnostic kit comprising a container comprising a composition according to claim 1, and/or a container comprising a composition according to claim 4.

7. The diagnostic kit according to claim 6, comprising a first container comprising the composition according to claim 1, and a second container comprising the composition according to claim 4.

8. The diagnostic kit according to claim 6, further comprising one or more reagents for detecting immunoglobulin (IG)/T cell receptor (TR) gene rearrangements.

9. The diagnostic kit according to claim 8, comprising a set of primers for amplicon-based next-generation sequencing (NGS) of IG/TR gene rearrangements.

10. The diagnostic kit according to claim 8, comprising primer sets for detecting one or more of the IG/TR gene rearrangements selected from the group consisting of IGH-VJ, IGH-DJ, IGK-VJ-Kde, TRB-VJ, TRB-DJ, TRG and TRD.

11. The diagnostic kit according to claim 9, comprising one or more of the primers selected from the primers shown in FIG. 5, preferably one or more of the primers selected from the primers shown in Table 3.

12. A set of primers for amplicon-based next-generation sequencing (NGS) of IG/TR gene rearrangements, comprising two or more of the primers selected from the primers shown in FIG. 5.

13. The set of primers according to claim 12, comprising two or more of the primers selected from the primers shown in Table 3.

14. The use of a composition according to claim 1, a kit according to claim 6, and/or a primer set according to claim 12 in an assay for detecting IG/TR gene rearrangements.

15. The use according to claim 14, wherein said assay is a clinical diagnostic assay, preferably an assay for detecting clonality, identifying minimal residual disease (MRD) markers and/or MRD monitoring and/or analyzing the (clonal) immune repertoire in a lymphoid malignancy.

16. An in vitro method for detecting IG/TR gene rearrangements in at least one biological sample using NGS, comprising the steps of sample preparation, PCR and/or library construction, sequencing and bioinformatics analysis, wherein the at least one biological sample is spiked with a composition according to claim 1, and/or wherein a composition according to claim 4 is run as a sample parallel to the at least one biological sample(s).

17. The method according to claim 16, wherein the at least one biological sample is a clinically relevant sample, preferably a sample for detection of clonality to support or exclude the diagnosis of malignant lymphoproliferation, or a sample taken for MRD marker identification or for MRD monitoring analysis or for (clonal) immune repertoire analysis.

18. The method according to claim 17, wherein at least part of the method is performed using a microfluidics device.

19. The method according to claim 18, wherein said microfluidics device comprises a centrifugal-microfluidic disk system, preferably wherein the disk comprises pre-stored reagents for automated and integrated DNA extraction, PCR and/or library generation.

20. The method according to claim 17, wherein the step of bioinformatic analysis comprises the use of a web-based, interactive application for pre-processing of raw data, primer sequence analysis, immunogenetic annotation, post-processing of results, analysis and use of the cIT-QC (including for marker quantification), analysis and use of the cPT-QC (including for comparison to pre-analyzed stored reference datasets), reporting of/access to/visualization of results.
